(19) United States
(12) Patent Application Publication
(10) Pub. No.: US 2017/0290944 A1
(43) Pub. Date: Oct. 12, 2017

(54) Promotion of Wound Healing

(71) Applicant: NORTHWESTERN UNIVERSITY, Evanston, IL (US)

(72) Inventor: Amy S. Paller, Wilmette, IL (US)

(21) Appl. No.: 15/494,641

(22) Filed: Apr. 24, 2017

Related U.S. Application Data
(63) Continuation of application No. 14/699,521, filed on Apr. 29, 2015, now abandoned.
Application No. 14/699,521 is a continuation of application No. 13/571,567, filed on Aug. 10, 2012, now abandoned.
(60) Provisional application No. 61/522,025, filed on Aug. 10, 2011.

(51) Int. Cl. A61L 15/32; A61L 15/44

(52) U.S. Cl. CPC A61L 15/32; A61L 15/44; A61L 2300/414; A61L 2300/45; A61L 2300/432

(57) ABSTRACT

The present invention provides compositions and methods that promote wound healing in a subject with a cutaneous injury. In particular, the present invention provides systemic and/or local administration of one or more compositions that cause ganglioside depletion (e.g., glucosylceramide synthase (GCS) inhibitors) for the treatment of cutaneous wounds.

### CROSS-REFERENCE TO RELATED APPLICATIONS

The present invention is a continuation of U.S. patent application Ser. No. 13/571,567, filed Aug. 10, 2012, which claims the benefit of U.S. Provisional Patent Application Ser. No. 61/522,025, filed Aug. 10, 2011, each of which is incorporated by reference in its entirety.

### STATEMENT REGARDING GOVERNMENT FUNDING

This invention was made with government support under grant number R01 AR044619 awarded by the National Institutes of Health. The government has certain rights in the invention.

### FIELD OF THE INVENTION

The present invention provides compositions and methods that promote wound healing in a subject with a cutaneous injury. In particular, the present invention provides systemic and/or local administration of one or more compositions that cause ganglioside depletion (e.g., glucosylceramide synthase (GCS) inhibitors) for the treatment of cutaneous wounds.

### BACKGROUND OF THE INVENTION

A wound is a type of injury in which skin is torn, cut or punctured (an open wound), or where blunt force trauma causes a contusion (a closed wound). Open wounds can be classified according to the object that caused the wound. Incisions, or incised wounds, are caused by a clean, sharp-edged object such as a knife, a razor or a glass splinter. Lacerations are irregular tear-like wounds caused by some blunt trauma. Lacerations and incisions may appear linear (regular) or stellate (irregular). The term laceration is commonly misused in reference to incisions. Abrasions are superficial wounds in which the topmost layer of the skin (the epidermis) is scraped off. Abrasions are often caused by a sliding fall onto a rough surface. Puncture wounds, caused by an object puncturing the skin, such as a nail or needle. Bacterial infection of wounds can impede the healing process and lead to life threatening complications. Anyone can develop a wound or wound-related infection; however, some people who may have poor healing abilities, like the elderly, because of declining immune system. Individuals who are malnourished or who do not eat right foods and lack vitamins, nutrients or have protein deficiency are at risk too. Those who are chronically ill, bedridden or non-ambulatory also have high risk factors as well as people who have undergone prolonged corticosteroid use or have been administered a potent immunosuppressive drug. Radiation therapy patients as well as diabetics, the obese and those that have had a stroke or some sort of peripheral vascular disease have slow or poor wound healing processes, and are more likely to develop some sort of wound infection.

### SUMMARY OF THE INVENTION

In some embodiments, the present invention provides methods of promoting wound healing comprising depleting gangliosides in a subject. In some embodiments, a ganglioside depletion agent (e.g., glucosylceramide synthase inhibitor) is administered to a subject to promote wound healing. In some embodiments, the present invention provides methods of promoting wound healing comprising administering one or more glucosylceramide synthase inhibitors to a subject with one or more cutaneous wounds. In some embodiments, the subject is diabetic (e.g., Type I diabetes, Type II diabetes, gestational diabetes, etc.). In some embodiments, one or more ganglioside precursors (e.g., GM3) are targeted to promote wound healing. In some embodiments, conversion of ganglioside precursors (e.g., GM3) into gangliosides is inhibited to promote wound healing. In some embodiments, the subject is not diabetic. In some embodiments, one or more cutaneous wounds comprise one or more of incisions, lacerations, abrasions, puncture wounds, and closed wounds (e.g., diabetic ulcers, such as a foot ulcer). In some embodiments, a glucosylceramide synthase inhibitor is selected from PDMP, D-threo-EtDO-P4, ((1R, 2R)-nonanoic acid[2-(2′,3′-dihydro-benzo [1,4] dioxin-6′-yl)-2-hydroxy-1-pyrrolidin-1-ylmethyl-ethyl]-amide-L-tartaric acid salt, AMP-DNM and analogues, homologues, and functional equivalents thereof. In some embodiments, a glucosylceramide synthase inhibitor is administered systemically. In some embodiments, a glucosylceramide synthase inhibitor is administered locally. In some embodiments, a glucosylceramide synthase inhibitor is administered topically. In some embodiments, administering a glucosylceramide synthase inhibitor accelerates the rate of wound repair (e.g., the wound heals twice as fast as without said inhibitor). In some embodiments, administering a glucosylceramide synthase inhibitor reduces the chance of said wound becoming infected. In some embodiments, a ganglioside depletion agent is administered systemically. In some embodiments, a ganglioside depletion agent is administered locally. In some embodiments, a ganglioside depletion agent is administered topically. In some embodiments, administering a ganglioside depletion agent accelerates the rate of wound repair (e.g., the wound heals at least 1.5×, 2×, 3×, 4×, 5×, 6×, 7×, 8×, 9×, or 10× as fast as without said inhibitor). In some embodiments, administering a ganglioside depletion agent reduces the chance of said wound becoming infected. In some embodiments, a composition that inhibits conversion of ganglioside precursors (e.g., GM3) into gangliosides is administered systemically. In some embodiments, a composition that inhibits conversion of ganglioside precursors (e.g., GM3) into gangliosides is administered locally. In some embodiments, a composition that inhibits conversion of ganglioside precursors (e.g., GM3) into gangliosides is administered topically. In some embodiments, administering a composition that inhibits conversion of ganglioside precursors (e.g., GM3) into gangliosides accelerates the rate of wound repair (e.g., the wound heals twice as fast as without said inhibitor). In some embodiments, administering a composition that inhibits conversion of ganglioside precursors (e.g., GM3) into gangliosides reduces the chance of said wound becoming infected.

In some embodiments, the present invention provides a composition for wound care comprising one or more ganglioside depletion agents (e.g., a glucosylceramide synthase inhibitors) and an application element. In some embodiments, the present invention provides a composition for wound care comprising one or more glucosylceramide synthase inhibitors and an application element. In some embodiments, a glucosylceramide synthase inhibitor is selected from PDMP, D-threo-EtDO-P4, ((1R, 2R)-nonanoic acid[2-(2′,3′-dihydro-benzo [1,4]dioxin-6′-yl)-2-hydroxy-1-pyrrolidin-1-ylmethyl-ethyl]-amide-L-tartaric acid salt, AMP-DNM and analogues, homologues, and functional equivalents thereof. In some embodiments, the application element is configured for topical application to a wound. In some embodiments, the application element comprises a liquid, cream, paste, salve, balm, or semi-solid. In some embodiments, the application element comprises a patch, wrap, or bandage. In some embodiments, a composition further comprises one or more additional wound care agents. In some embodiments, wound care agents are selected from antiseptic, antibiotic, local anesthetic, anti-inflammatory, pain reliever, etc.

### Definitions

As used herein, the term “subject” refers to any animal (e.g., a mammal), including, but not limited to, humans, non-human primates, rodents, and the like, which is to be the recipient of compositions and methods that may be provided by the present invention. Typically, the terms “subject” and “patient” are used interchangeably herein in reference to a human subject.

### DESCRIPTION OF THE FIGURES

FIG. 1 shows a schematic of the ganglioside synthesis pathway. GM3, precursor for complex gangliosides, is made from lactosylCer (LacCer) by GM3 synthase (GM3S). Inhibition of glucosylceramide (GlcCer) synthase (GCS) depletes GM3, LacCer and GlcCer, while knockdown of GM3S specifically depletes GM3 and downstream gangliosides. Newer GCS inhibitors (C9, EtDOP4) do not cause Cer accumulation (Lee et al. J Biol Chem 1999; 274:14662-9; Natoli et al. Nat Med 2010; 16:788-92; herein incorporated by reference in their entireties) due to shunting to 1-acylCer. Blockade of GD3 and GM2/GD2 synthases increases GM3.

FIGS. 2A and 2B show graphs demonstrating that diabetic mouse skin shows increased GM3S and GM3 expression. A. GM3S expression was measured by qPCR and normalized to β-actin. B. After fat removal, skin gangliosides were extracted with chloroform/methanol (1/1, v/v) from ob/ob, WT and GM3S knockout mice. GM3 was purified by a C18 column, and detected by thin layer chromatography (TLC) immunostaining (Wang et al. J Biol Chem 2002; 277:47028-34; Wang et al. J Biol Chem 2001; 276:8436-44; herein incorporated by reference in their entireties). RD=regular diet; HFD=high fat diet; Std=GM3 standard.

FIGS. 3A-G show graphs and images demonstrating that both chronic exposure to cytokine TNF-α and increased ambient glucose increase GM3S and GM3 in HEKs. A. HEKs were treated for 96 h with 0-100 pM TNF-α in complete M154 medium and GM3S expression determined by qPCR. B. GM3 was quantified by flow cytometry with anti-GM3 antibody after 0-100 pM TNF-α for 96 h. C, D. HEKs were treated with 12 mM supplemental glucose (C) or in M154 medium alone (D) for 72 h and GM3 was detected with anti-GM3 antibody by immunofluorescence. Flow cytometry studies show that TNF-α increases membrane-based GM3 expression by 10-fold and glucose by 73%. E, F. Treating HEKs with 1 μM C9 for 72 h prevents GM3 expression in M154 medium (E) and glucose-supplemented medium (F). Quantification by flow cytometry shows a 93% reduction in GM3 expression by C9. C9 treatment does not increase ceramides as detected with anti-C2 Cer antibody by TLC immunostaining (G) and ELISA.

FIGS. 4A-C show images demonstrating uptake of siRNA and DNA in ˜100% of HEKs. A. Cy3-labeled siRNA-Au NPs. B. Cy5-labeled DNA-Au NPs. DAPI, blue; actin, green. C. 24 h after topical application to mouse skin of 25 nM siRNA-Au NPs in Aquaphor, uptake (pink) in this overlap image of Cy3-siRNA-Au NPs and DAPI is seen in >90% of cells, including basal KCs. Hair and stratum corneum autofluoresce. Bar=20 μm.

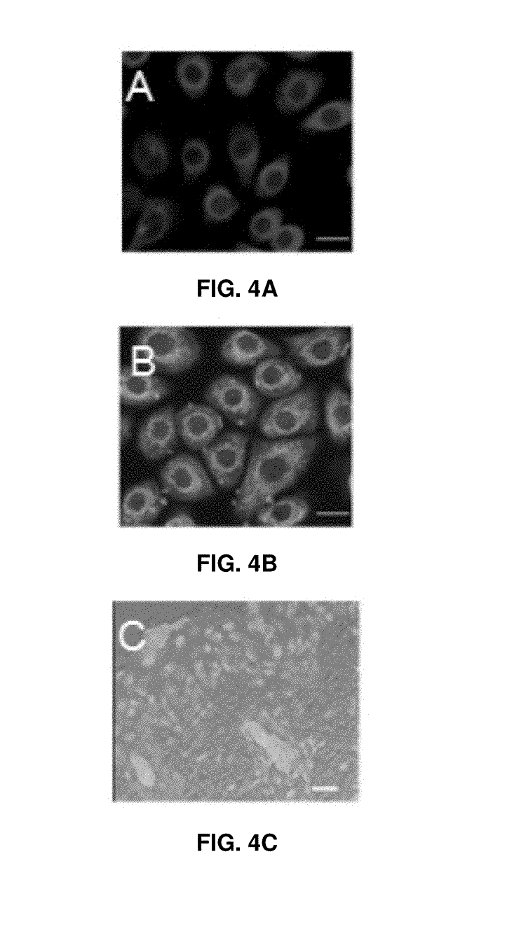

FIGS. 5A-D show images demonstrating the efficacy of oligonucleotide-Au NPs in modulating GM3. Changes in GM3 expression by immunofluorescence with anti-GM3 antibody show efficacy of siRNA- and DNA-Au NPs with 60-72 h incubation. A. No diminution in GM3 expression with scrambled siRNA-Au NPs. B. Elimination of GM3 by GM3S siRNA-Au NPs. C. Effects of sense DNA-Au NPs did not differ from vehicle treatment. D. Increase in GM3 expression with hybrid GD3S GM2/GD2S DNA-Au NP treatment. GM3 clustering at the membrane, suggesting accumulation of GM3 in lipid rafts, is seen in the magnified inset (arrows). A, B=routine immunofluorescence; C, D and inset of D=confocal microscopy. Bar=14 μm.

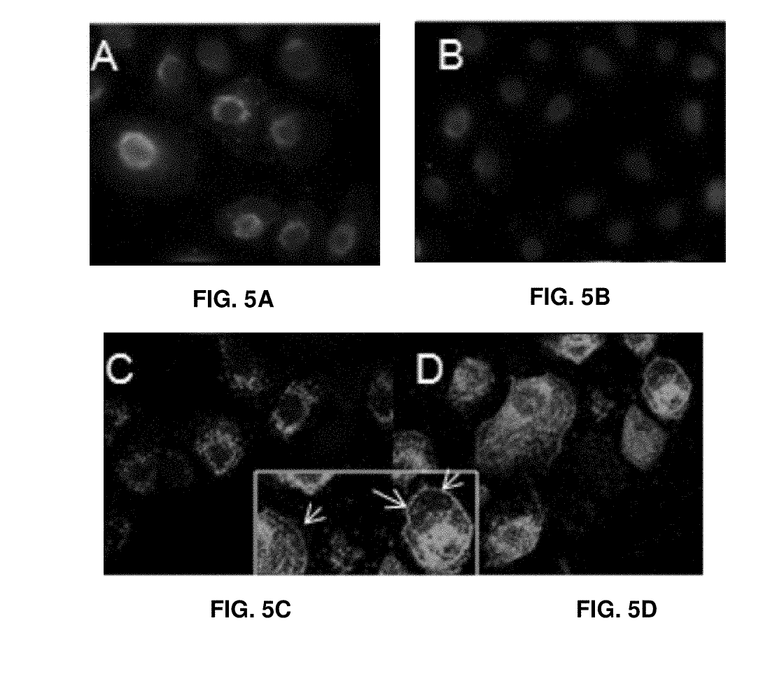

FIGS. 6A and 6B show (A) images of GM3S knockout mice (KO) and wildtype (WT) littermates on a regular diet (RD) or 45% high fat diet (HFD) for 10 weeks (beginning at weaning) were wounded with a 5 mm punch and fitted with a splint to minimize wound contracture. Clinical differences in wound healing were seen by 3 days between WTHFD and KOHFD mice. (B) The epidermal gap is measured in histological sections as the distance between the leading edges of epidermis.

FIGS. 7A and 7B shows results of experiments demonstrating ganglioside depletion in GM3S−/− mice reverses glucose-induced inhibition of proliferation and migration. A. KO and WT mouse KCs, grown in CNT07 medium, were treated with and without 12 mM glucose (Glu) for 72 h, and then plated into 96-well plates. Proliferation was measured by WST assay (Clontech) in the continued presence or absence of Glu. B. Mouse KCs were treated as above, then plated at confluence into Col I-coated 24-well plates in the presence of mitomycin C. Two hours after plating, a scratch was made and migration monitored. Migration was decreased by Glu, but was faster in the KO cells treated with Glu than with vehicle.

FIGS. 8A-G show the results of experiments demonstrating that ganglioside depletion by C9 in HEKs reverses glucose-induced inhibition of proliferation and migration, particularly in the presence of insulin and IGF-1. A. HEKs were treated as described in FIG. 7 for mouse KCs and proliferation was measured. By 5 days, proliferation was greatest in cells treated with Glu and C9, and least in cells treated only with Glu. B. HEKs were treated and scratch assays performed as in FIG. 7. Migration was decreased by Glu, but even faster in the HEKs treated with C9+Glu than in vehicle-treated HEKs. C. HEKs were treated with Glu and/or C9, but starved during the last 18 h. Cells were plated on the top of Col I-coated Boyden inserts in supplement-free medium, while the lower level was filled with the same medium with and without insulin (5 μg/ml) or IGF-1 (100 ng/ml) (Haase et al. J Cell Sci 2003; 116:3227-38; herein incorporated by reference in its entirety). After 18 h, HEKs on the bottom surface were stained and counted. C9 treatment increased chemotaxis of HEKs exposed to supplemental Glu, esp. in the presence of insulin or IGF-1. D-G. After their incubation in Glu and/or C9, HEKs were plated onto Col I-coated glass dishes and, 2 h later, single cells were imaged every 2 mins over a 2 h period using a Biostation phase contrast microscope (Nikon). Most Glu-treated cells were round and surrounded by ruffling, but had no lamellipodia, whereas the addition of C9 and, even more so C9 and insulin/IGF-1, increased cell polarity (D, E). Cells barely moved with Glu treatment, but at 2 h both net displacement (reflecting persistence) and total distance (reflecting velocity) were significantly increased by Glu+C9 alone and the combination of Glu+C9/insulin or C9/IGF-1 (F). Vector maps constructed with 10 representative tracks (G) further show reversal of the impact of Glu with C9 alone (which was approximately the same as with growth factors).

FIGS. 9A and 9B show images of immunoblots demonstrating that high glucose and increased GM3 increase p-cofilin expression. HEKs were treated for 72 h with or without Glu, C9, Glu+C9 (A), or with sense DNA-Au NPs or the hybrid GM2/GD2S and GD3S antisense-Au NPs (B). Immunoblotting of cell lysates detected p-cofilin8 (Ser3), cofilin and, as a loading control, GAPDH.

FIGS. 10A-D show images of immunoblots demonstrating that GM3 suppresses and ganglioside depletion increases insulin signaling triggered by either insulin or IGF-1R. A, B. HEKs were treated for 72-96 h with or without either GCS inhibition (100 pM EtDOP4; similar results with C9), 100 pM TNF-α, the combination, or GM3 50 μM. Cells were starved of growth factors overnight (with treatment continued), then treated for 5-60 mins (15 mins shown) with or without insulin 5 μg/ml. Cells lysates were immunoprecipitated with anti-IR polyclonal antibody and blotted with monoclonal anti-IR, PY-20, or as a loading control, actin. Membrane lysates were immunoprecipitated with IR and immunoblotted with anti-IR (top), PY-20 (second row), anti-IRS-1 (3rd row in B), or to control for loading, actin (bottom). TNF-α and GM3 suppresses IR signaling; ganglioside depletion rescues the suppression of TNF-α of IR phosphorylation. Expression of IR was not affected. C, D. HEKs were treated for 72 h without or with Glu and/or C9 (C) or with antisense GM2/GD2S+GD3S DNA-Au NPs (either as 1 nM of hybrid molecule (left) or 500 nM each of both antisense DNA-Au NPs (right) and sense DNA-Au NPs as an additional control. After starvation, HEKs were treated for 5-60 min with IGF-1 100 ng/ml (30 min shown) or insulin 5 μg/ml. PY20 antibody was used to immunoprecipitate tyr-phosphorylated proteins, and anti-IGF-1R to detect p-IGF-1R (and IGF-1R in whole lysates). Glucose suppresses p-IGF-1R, even in the presence of IGF-1; C9 phosphorylates IGF-1R, including in the presence of supplemental glucose.

FIG. 11 shows a graph demonstrating that GM3 synthase and GM3 ganglioside are increased in diabetic mouse skin. Expression of GM3S in skin devoid of adipose tissue was detected by qRT-PCR (A) and ganglioside GM3 by thin layer chromatography (TLC) immunostaining with anti-GM3 antibody (B). The absence of GM3 in TLCs from GM3S−/− mouse skin is apparent regardless of diet.

FIGS. 12A-D show images of wounds and graphs of wound area demonstrating that ganglioside depletion accelerates wound healing in DIO mice. GM3S^{-/-} mice and wildtype (WT) littermates were fed a regular diet (RD) or 42% high fat diet (HFD) for 9-10 wks before wounding. Wounds were made bilaterally on the backs of GM3S^{-/-} mice and littermate (GM3S^{+/+} WT) controls, and mice were fitted with a splint to minimize wound contracture (Galiano et al. 2004. Wound Repair Regen 12:485-492; herein incorporated by reference in its entirety). (A) Representative wounds are shown before splinting at baseline and after splint removal at days 3 through 7. (B) The open wound area was determined by computerized measurements of photographs and expressed as a percentage of the original wound area. (C, D) At 3, 5, and 7 days after wounding, the entire wound was harvested for histological evaluation. Epidermal gap as the maximal distance between leading edges of epidermis (C) and granulation tissue area (D) were measured by computerized morphometry. By 3 days, the epidermal gap was greater for WT HFD vs. all other mice and conditions.

FIGS. 13A-C show ganglioside depletion reverses glucose-induced inhibition of proliferation and in vitro wound healing. (A) Mouse KCs were cultured in standard medium (8 mM glucose) or medium supplemented with 12 mM glucose treated for 72 h, and then plated at confluence onto collagen I-coated 24-well plates in the presence of mitomycin C to prevent proliferation. Two hours after plating, a scratch was made and migration was monitored photographically during the subsequent 60 h in the continued presence or absence of glucose. (B) Quantification of wound healing shown in (A) by computerized measurements of the unfilled gap. (C) Proliferation of mouse KCs was measured by WST assay in standard or glucose-supplemented medium.

FIG. 14 shows images of immunofluorescence and immunoblotting experiments demonstrating that exposure to increased glucose increases GM3 and GM3S expression in KCs. WT and GM3S^{-/-} mouse KCs were treated with 12 mM supplemental glucose for 72 h to simulate hyperglycemia (right) or in standard medium alone (left). (A) GM3 expression was detected with anti-GM3 antibody by immunofluorescence staining. (B) Immunoblotting demonstrates that the increase in GM3 is associated with 4-fold increased expression of GM3S.

FIGS. 15A-F show the results of live cell imaging studies demonstrating that ganglioside depletion reverses glucose-induced suppression of lamellipodium formation, KC velocity and net displacement. Live cell imaging studies of >100 single migrating mouse KCs from each mouse or condition were performed to assess cell shape (A), number of lamellipodia (B), velocity (C), persistence (D), and net displacement (E). Vector maps were generated and the tracked migration of the 10 displayed KCs are representative of 100 cells in each group (F). Cells were tracked every two mins for 2 h. Data are shown as means±S.D. Note the loss of lamellipodia in 70.9% of glucose-treated WT mouse KCs.

FIGS. 16A-D show depletion of GM3 activates IR/IGF-1R signaling. GM3S^{-/-} and control littermate GM3S^{+/+} WT KCs were treated with vehicle control (Ctrl), insulin (Ins, 5 μg/mL) or IGF-1 (100 ng/mL) in the absence or presence of 12 mM supplemental glucose. Phosphorylation of IR (A) and IGF-1R (B) (top rows) was detected by antibodies against p-IR and p-IGF-1R, respectively, in whole cell lysates. GAPDH was detected to assure even loading (bottom rows). Densitometry was performed to quantify bands using the NIH ImageJ program. Band density values of each group were normalized with their loading (GAPDH measurement) controls. (C) Changes in receptor phosphorylation as detected in (A) and (B) by direct antibody measurement were confirmed by immunoprecipitation of IR or IGF-1R and probing with PY-20 antibody. (D) Receptor expression was examined by qRT-PCR as described in “Materials and Methods”.

FIGS. 17A-C show GM3 depletion promotes the association of IRS-1 with IR and IGF-1R receptors and activates AKT signaling. GM3S^{-/-} and control littermate GM3S^{+/+} WT KCs were treated with insulin (5 μg/mL) or IGF-1 (100 ng/mL) in the absence or presence of 12 mM supplemental glucose. (A) IR and IGF-1R were immunoprecipitated from whole cell lysates (top rows) to assess their association with IRS-1 (middle rows) and with each other (bottom rows). (B) IRS-1 expression was detected in whole cell lysates with anti-IRS-1 antibody (top row) and equal loading confirmed by detection of GAPDH (2nd row); IRS-1 was immunoprecipitated from whole cell lysates (3rd row) and tyrosine phosphorylated IRS-1 (p-IRS-1) was detected with PY-20 antibody (bottom row). (D) AKT and AKT phosphorylation at Thr308 and Ser473 sites was detected in whole cell lysates with their respective antibodies.

### DETAILED DESCRIPTION OF THE INVENTION

The present invention provides compositions and methods that promote wound healing in a subject with a cutaneous injury. In particular, the present invention provides systemic and/or local administration of one or more compositions that cause ganglioside depletion (e.g., glucosylceramide synthase (GCS) inhibitors) for the treatment of cutaneous wounds. In some embodiments, the present invention provides systemic and/or local administration of glucosylceramide synthase (GCS; a.k.a.: UDP-glucose:ceramide glucosyltransferase, UDP-glucose:N-acylsphingosine D-glucosyltransferase, EC 2.4.1.80) inhibitors for the treatment of wounds (e.g., cutaneous wounds). In some embodiments, the wound healing is promoted though ganglioside depletion. In some embodiments, the wound healing is promoted by inhibiting the conversion of ganglioside precursors (e.g., GM3S) into gangliosides. In some embodiments, the present invention provides systemic, local, and/or topical administration of GCS inhibitors to a wound or a subject having one or more wounds in order to promote (e.g., advance, accelerate, and/or aid in) wound healing and the prevention of infection. In some embodiments, GCS inhibitors are administered (e.g., systemic, local, and/or topical administration) to a subject suffering from diabetes to promote wound healing. In some embodiments, GCS inhibitors are administered (e.g., systemic, local, and/or topical administration) to a subject who is not suffering from diabetes to promote wound healing.

Chronic cutaneous ulcerations are a major health issue among individuals with diabetes, a condition of insulin signaling resistance that afflicts almost 24 million U.S. adults (CDC. National diabetes fact sheet: general information and national estimates on diabetes in the United States, 2005. Atlanta (Ga.): US Department of Health and Human Services, Centers for Disease Control and Prevention; herein incorporated by reference in its entirety) and is related to obesity. Poor wound healing in diabetics has been attributed predominantly to the hyperglycemic milieu, insulin insensitivity, chronic exposure to cytokines including TNF-α (Hotamisligil et al. Proc Natl Acad Sci USA 1994; 91:4854-8; Goren et al. Am J Pathol 2006; 168:765-77; herein incorporated by reference in their entireties) and hypoxia from vascular insufficiency. Management of diabetic wounds represents a huge economic burden. Wound healing is a complex process that requires proliferation and migration of keratinocytes (KCs) to re-epithelialize the wound. Extracellular matrix (ECM) components, among them collagen I and fibronectin, activate integrin signaling and initiate cell migration, while growth factors optimize migration (Li et al. J Invest Dermatol 2006; 126:2096-105; herein incorporated by reference in its entirety). The most potent stimulants of KC migration at the wound site are ligands for epidermal growth factor receptor (EGFR), and insulin and insulin-like growth factor 1 (IGF-1), drivers of insulin signaling which are both able to activate receptors for insulin (IR) and IGF-1 (IGF-1R) (Li et al. J Invest Dermatol 2006; 126:2096-105; Ando et al. J Invest Dermatol 1993; 100:633-9; Stachelscheid et al. Embo J 2008; 27:2091-101; herein incorporated by reference in their entireties). Experiments were conducted during the development of the present invention that indicate that gangliosides play a role in the ability of skin cells to migrate and proliferate, including in response to stimulants of EGFR, IR and IGF-1R. Data also indicate a critical role for gangliosides in mediating insulin resistance in KCs. Experiments conducted during development of the present invention indicate that depletion of ganglioside promote wound healing (e.g., including erosive, ulcerative, and blistering disorders) and a wide variety of other disorders of skin in which proliferation is increased, resulting in skin thickening.

Glucosylceramide synthase (GCS) inhibitors have been tested in mice and in early human trials as pharmacologic agents to reverse the manifestations of type II diabetes. D-threo-1-phenyl-2-decanoylamino-3-morpholinopropanol (PDMP) is the prototypic GCS inhibitor, 1^{st} described in 1980. Since that time, a series of homologues have been described that have greater specificity and activity than PDMP against GCS. D-threo-EtDO-P4 is a variant that has been shown to decrease the accumulation of globotriaosylceramide in patients with Fabry disease. An analog of EtDOP4 (Genz-123346), contains 7 carbon chains less than the D-threo-EtDO-P4. Although Genz-123346 is somewhat less potent that P4 at inhibiting GCS, it has a better metabolic and tolerability profile in animals. (N-(5′-adamantane-1′-yl-methoxy)-pentyl-1-deoxynojirimycin (AMP-DNM) is a GCS inhibitor that also enhances insulin sensitivity that is an iminosugar derivative.

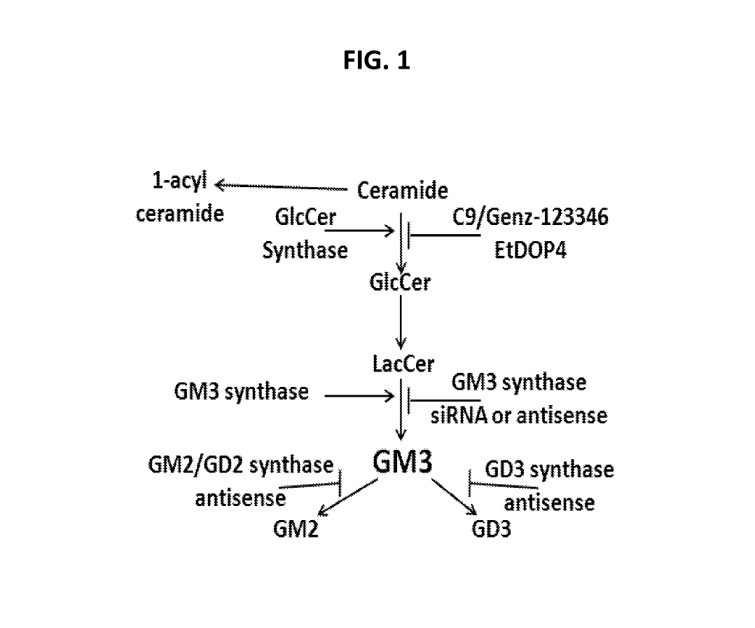

GM3 is the predominant sialic acid-containing glycosphingolipid of both KC and adipocyte membranes, and the precursor for more complex gangliosides (SEE FIG. 1). Experiments conducted during development of the present invention in adipose tissue, muscle and liver suggest that GM3 mediates cytokine-induced insulin resistance. Cytokines associated with obesity, particularly TNF-α, increase GM3 synthase (GM3S) and GM3 expression in adipocytes, myocytes and hepatocytes (Tagami et al. J Biol Chem 2002; 277:3085-92; Memon et al. J Biol Chem 1999; 274:19707-13, herein incorporated by reference in their entireties).

GM3 is markedly increased in the adipose and muscle tissue of diabetic fatty (fa/fa) rats and ob/ob mice (Tagami et al. J Biol Chem 2002; 277:3085-92; herein incorporated by reference in its entirety). In adipocytes, GM3 suppresses Tyr phosphorylation of the insulin receptor-β(IR) and IRS-1, decreasing glucose uptake (Tagami et al. J Biol Chem 2002; 277:3085-92; herein incorporated by reference in its entirety). GM3S knockout mice on a high-fat diet show increased insulin sensitivity in muscle and adipose tissue (Yamashita et al. Proc Natl Acad Sci USA 2003; 100:3445-9, herein incorporated by reference in its entirety). Glucosylceramide (GlcCer) synthase (GCS) inhibitors reverse TNF-α-induced insulin resistance in adipocytes and hepatocytes, and improve insulin sensitivity in adipose tissue, muscle and liver of diet-induced obese (DIO) diabetic mouse and rat models (Tagami et al. J Biol Chem 2002; 277:3085-92; Zhao et al. Diabetes 2007; 56:1210-8; Aerts et al. Diabetes 2007; 56:1341-9; Yew et al. PLoS One 2010; 5:e11239; herein incorporated by reference in their entireties). Data invention indicte that glycosphingolipids, and especially GM3, are important drivers of insulin resistance, downstream of cytokine activity. As such, depletion of gangliosides represents a new approach to increasing insulin sensitivity.

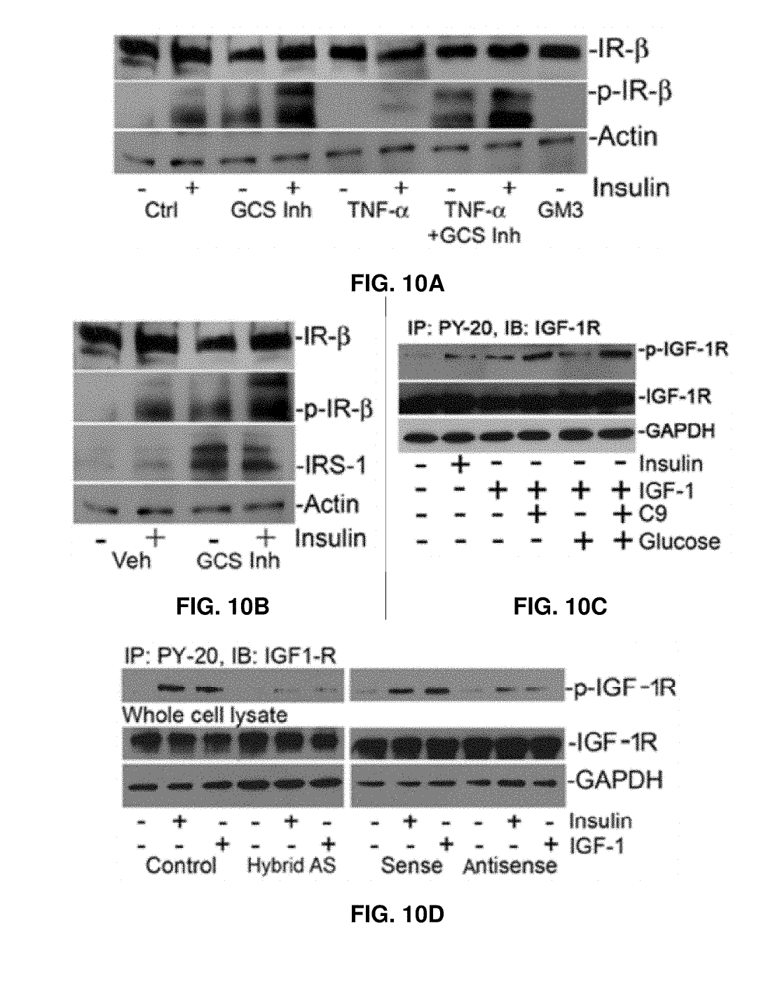

Experiments conducted during development of embodiments of the present invention indicate that increased GM3 alone (e.g., biochemically or by antisense blockade of its metabolizing enzymes) is sufficient to suppress IR and IGF-1R activity (SEE FIG. 10). In addition to GM3, GT1b (a trisialylated product of GM3) is the only other ganglioside that blocks KC migration; however, GT1b interacts with ac5 integrin to inhibit KC migration specifically on fibronectin (FN) (Paller et al. J Invest Dermatol 1995; 105:237-42; herein incorporated by reference in its entirety). Ganglioside depletion increases insulin-induced activation of IR (SEE FIG. 10A) and supplementation with GM3 suppresses it, as has been shown for adipocytes. Co-immunoprecipitation of IRS-1 and IR by ganglioside depletion is further evidence of activated insulin signaling (SEE FIG. 10B). Ganglioside depletion also promotes insulin- and IGF-1-induced activation of IGF-1R (SEE FIG. 10C). Supplemental glucose decreases phosphorylation of KC IGF-1R, but not IR, regardless of stimulation with insulin or IGF-1 (Spravchikov. Diabetes 2001; 50:1627-35; herein incorporated by reference in its entirety). C9 reverses the glucose-induced suppression of IGF-1R activation (SEE FIG. 10C). Increasing GM3 through antisense Au—NP blockade of downstream metabolism also suppresses phosphorylation of IGF-1R without affecting its expression (SEE FIG. 10D).

Experiments were conducted during development of embodiments of the present invention that demonstrate that GM3S depletion fully reverses the impairment in wound healing in a diet-induced diabetic mouse model, despite mouse obesity and only partial improvement in systemic glucose homeostasis. Although various mechanisms for the above action and effect are discussed herein, the present invention is not limited to any particular mechanism of action and an understanding of the mechanism of action is not necessary to practice the present invention. In some embodiments, increases in migration and/or proliferation of KCs contribute to the accelerated healing. Furthermore, a direct effect of GM3S depletion on KC motility and proliferation through activation of both insulin and IGF-1 receptors has been demonstrated, particularly when KCs are made “diabetic” through exposure to increased glucose concentration. The increases in KC migration and proliferation with GM3 depletion and hyperglycemia in vitro paralleled the increases in BrdU labeling and length of the migrating epidermal tongue in the wounds of GM3S^{-/-} obese mice. These observations indicate a central role for ganglioside GM3 in the wound healing defect of obesity-related diabetes, and raise the possibility of topical treatment to reduce gangliosides as a future therapy for wounds (e.g., in diabetic subjects).

Hyperglycemic medium inhibits human KC migration and mouse KC proliferation in a concentration-dependent manner Lan et al. 2008. Br J Dermatol; Spravchikov et al. 2001. Diabetes 50:1627-1635; Terashi et al. 2005. Int Wound J 2:298-304; herein incorporated by reference in their entireties). Through live cell imaging studies, Experiments conducted during development of embodiments of the present invention dissected the impact of hyperglycemia on mouse KC shape and the formation of lamellipodia, sheet-like cytoplasmic extensions that promote cell movement. Cell migration virtually ceased in the presence of excess glucose, in concert with KC rounding and loss of lamellipodia. In GM3S^{-/-} KCs exposed to hyperglycemia, cell migration did not cease, but rather increased in cell velocity, displacement, and persistence. Instead of rounding as in WT cells, GM3S^{-/-} cells showed an 8-fold increase in the presence of a single lamellipodium for directional migration; the majority of these GM3S^{-/-} KCs showed a large sail-shaped lamellipodium that was only occasionally seen in WT KCs, but is seen in highly motile cells.

The improved insulin sensitivity of ganglioside-depleted adipocytes has linked to increased adipocyte IR autophosphorylation (Tagami et al. 2002. J Biol Chem 277:3085-3092; Kabayama et al. 2005. Glycobiology 15:21-29; herein incorporated by reference in their entireties). Experiments conducted during development of embodiments of the present invention demonstrated that the increased responses of IR to IGF-1 and of IGF-1R to its ligands in GM3S^{-/-} KC are even more dramatic than the increased IR autophosphorylation. Indeed, IGF-1, which is produced by wound dermal fibroblasts, accelerates in vitro (Haase et al. 2003. J Cell Sci 116:3227-3238; herein incorporated by reference in its entirety) and in vivo (Jeschke et al. 2004. Am J Physiol Regul Integr Comp Physiol 286:R958-966; Jyung et al. 1994. Surgery 115:233-239; Semenova et al. 2008. Am J Pathol 173:1295-1310; herein incorporated by reference in their entireties) wound healing and regulates KC shape, promoting membrane protrusion. Experiments conducted during development of embodiments of the present invention demonstrate that GM3S prevents glucose-induced IGF-1R inhibition, in addition to reversing glucose-induced IR autophosphorylation in KCs.

Experiments were conducted during development of embodiments of the present invention which demonstrate that exposure to hyperglycemia leads to GM3 accumulation, at least in part through increased GM3S expression, suggests that GM3 accumulation plays a role in glucose-induced insulin resistance in KCs; although the present invention is not limited to any particular mechanism of action and an understanding of the mechanism of action is not necessary to practice the present invention. Findings indicate that glucose also increases both GM3 and GM3S expression in mouse KCs implicates a positive feedback loop that perpetuates the insulin resistance, with chronic exposure to low levels of TNF-α initiating the insulin resistance, at least in part through increasing GM3S, and the resulting hyperglycemia further increasing GM3 levels to sustain the resistance. While the most dramatic increases in migration and proliferation of GM3S^{-/-} KCs in comparison with WT KCs occurred in hyperglycemic medium, significant increases were also detected in standard medium in vitro. The lack of translation of this finding into accelerated re-epithelialization of wounds in mice on a RD can be explained by the relatively small size of the mouse wounds, which may have been insufficiently sensitive to detect the inhibitory effect of ganglioside depletion without hyperglycemia.

Experiments conducted during development of embodiments of the present invention indicate a role for GM3 accumulation in the impaired wound healing of diabetic skin, both through a direct inhibition of insulin and IGF-1 receptor activation and by preventing glucose-induced insulin resistance. In some embodiments, biochemical and/or genetic ganglioside depletion provides a therapeutic approach to wound healing (e.g., diabetic wound healing), given its stimulatory effect on keratinocyte proliferation and migration.

In some embodiments, the present invention provides administration of gangliside depletion agents, GCS inhibitors, and/or inhibitors of conversion of ganglioside precursors into gangliosides to a subject for the promotion of wound healing. The present invention is not limited to any particular mechanism of action and an understanding of the mechanism of action is not necessary to practice the present invention. Any compounds (e.g., small molecules, proteins, peptide, carbohydrates, lipids, complexes, organic molecules, derivatives thereof, and combinations thereof) suitable for depletion of gangliosides find use in the present invention. Any compounds (e.g., small molecules, proteins, peptide, carbohydrates, lipids, complexes, organic molecules, derivatives thereof, and combinations thereof) suitable for inhibition of GCS activity or expression find use in the present invention. In some embodiments, GCS inhibitors and/or ganglioside depletion agents are administered to a subject suffering from diabetes (e.g., type I, type II) to promote wound healing (e.g., independent from diabetic treatment). In some embodiments, GCS inhibitors and/or ganglioside depletion agents are administered to a non-diabetic subject. In some embodiments, GCS inhibitors and/or ganglioside depletion agents are administered for the purpose of wound care, and not for treatment of diabetes.

In some embodiments, GCS inhibitors are co-administered with one or more other and/or ganglioside depletion agents. In some embodiments, GCS inhibitors and/or ganglioside depletion agents are co-administered with other treatments for diabetes. In some embodiments, GCS inhibitors and/or ganglioside depletion agents are co-administered with other treatments for wound care. In some embodiments, GCS inhibitors and/or ganglioside depletion agents are co-administered with one or more of antiseptic, antibiotic, local anesthetic, anti-inflammatory, pain reliever, etc. In some embodiments, GCS inhibitors and/or ganglioside depletion agents are configured for administration through any suitable route including: capsule, pill, injection, cream, ointment, lotion, slave, balm, suppository, solution, elixir, syrup, suspension, cream, lozenge, paste, spray, patch, bandage, wrap, etc. In some embodiments, GCS inhibitors and/or ganglioside depletion agents and other active or inactive agents are administered to a subject by any of the routes conventionally used for drug administration, for example they may be adapted for oral (including buccal, sublingual), topical (including transdermal), nasal (including inhalation), rectal, vaginal or parenteral (including subcutaneous, intramuscular, intravenous or intradermal) administration to mammals including humans. The most suitable route for administration in any given case will depend on the particular composition, the subject, the nature and severity of the wound, the application, and the desired effect. Such compositions may be prepared by any method known in the art of pharmacy, for example by bringing into association the active ingredient with the carrier(s), excipient(s) and/or diluent(s).

Compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions. Tablets and capsules for oral administration may be in unit dose presentation form and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Compositions adapted for topical administration may be formulated as ointments, creams, suspensions, lotions, powders, solutions, pastes, gels, impregnated dressings (e.g. bandages, wraps, patches, etc.), sprays, aerosols or oils and may contain appropriate conventional additives such as preservatives, solvents to assist drug penetration and emollients in ointments and creams. In some embodiment GCS inhibitors and/or ganglioside depletion agents and other active or inactive agents are employed with either a paraffinic or a water-miscible ointment base, or formulated in a cream with an oil-in-water cream base or a water-in-oil base. The composition may also contain compatible conventional carriers, such as cream or ointment bases and ethanol or oleyl alcohol for lotions. Compositions adapted for transdermal administration may be presented as discrete patches intended to remain in intimate contact with the epidermis of the recipient (e.g. at the wound site, adjacent to the wound site, atop the wound, etc.) for a prolonged period of time (e.g., 1 minute . . . 5 minutes . . . 10 minutes . . . 1 hour . . . 2 hours . . . 5 hours . . . 12 hours . . . 24 hours . . . 1 week, etc.). For example, the active ingredient may be delivered from the patch by iontophoresis as generally described in Pharmaceutical Research, 3(6), 318, (1986).

In some embodiments, appropriate dose of one or more GCS inhibitors and/or ganglioside depletion agents, as well as other active agents (e.g., antiseptic, anti-inflammatory, anesthetic (e.g., local, topical, etc.), antibiotic, antifungal, antibacterial, etc.) is determined by user and/or clinician based on the wound, subject, route of application, etc.

### EXPERIMENTAL

The following examples are provided in order to demonstrate and further illustrate certain preferred embodiments and aspects of the present invention and are not to be construed as limiting the scope thereof.

### Example 1

### GM3 Mediates Both Hyperglycemia-Induced and Cytokine-Induced Insulin Resistance in Human Epidermal KCs (HEKs), while Ganglioside Depletion Promotes Diabetic Wound Healing in Obese Mice

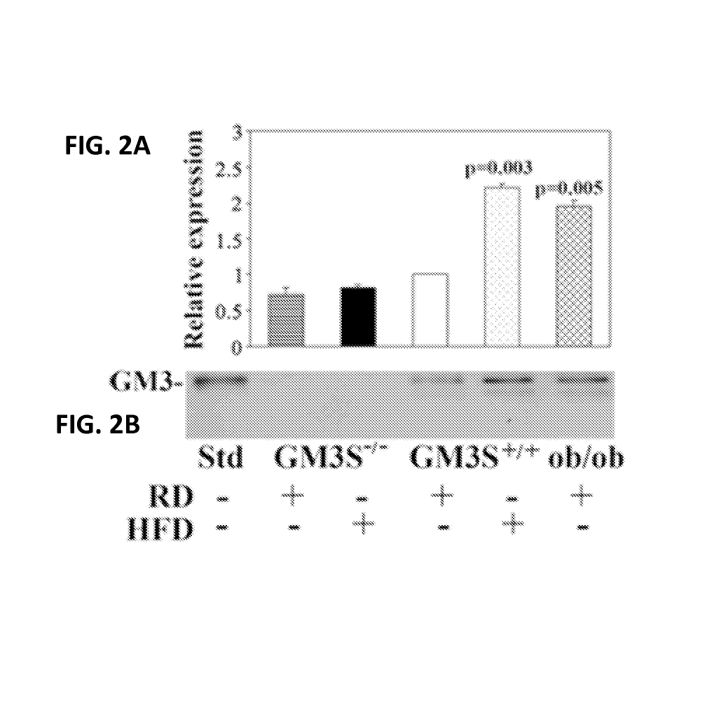

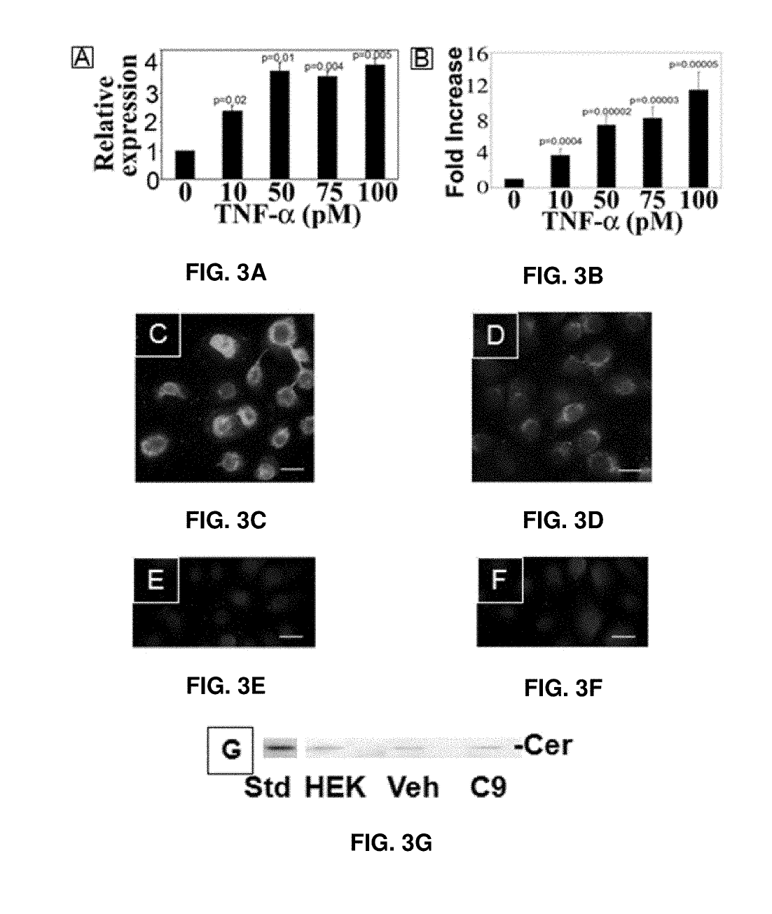

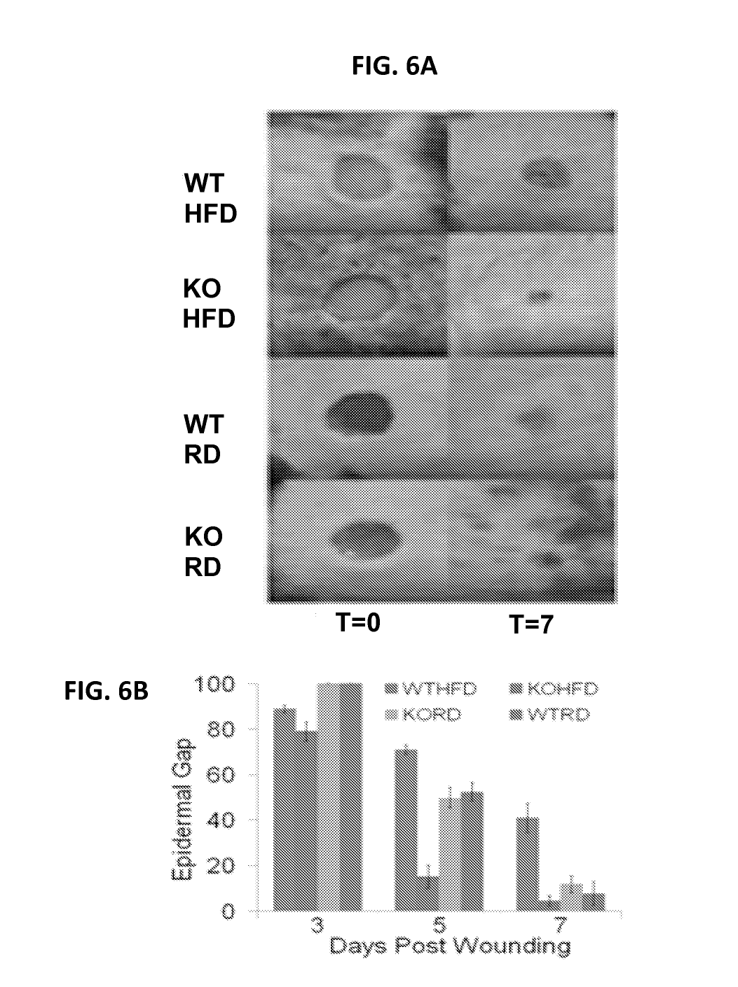

Experiments conducted during the development of embodiments of the present invention demonstrate that the skin of diabetic mice (DIO and ob/ob), as their adipose tissue and muscle, shows increased expression of GM3S (SEE FIG. 2, top) and GM3 (SEE FIG. 2, bottom), suggesting that GM3 could directly suppress insulin signaling in skin. Data also demonstrate that ganglioside depletion by knockout of GM3S promotes wound healing in DIO mice, markedly accelerating epidermal wound closure (SEE FIG. 6). GM3 is increased in HEKs by chronic exposure to either low concentrations of TNF-α (SEE FIG. 3A,B), as occurs in obesity, or increased glucose, simulating hyperglycemia (SEE FIG. 3C,D), indicating that GM3 is a mediator of both cytokine- and hyperglycemia-induced insulin resistance. Experiments conducted during the development of embodiments of the present invention demonstrate that that depletion of ganglioside both genetically (obese GM3S knockout mouse KCs) and biochemically (C9-treated HEKs) promotes KC proliferation and migration (SEE FIGS. 6-10). Ganglioside depletion directly affects KCs in accelerating wound healing. Ganglioside depletion increases IR activation, as in adipocytes, and ganglioside depletion activates IGF-1R; increased GM3 suppresses ligand-induced activation of both IR and IGF-1R, implicating GM3 itself as the mediator of insulin resistance. The importance of IGF-1R activation in diabetic skin is further demonstrated by experiments conducted during development of embodiments of the present invention demonstrating that increases in glucose impact activation of IGF-1R, but not IR, regardless of whether triggered by insulin or IGF-1, and that C9 is able to reverse the suppression of IGF-1R phosphorylation (SEE FIG. 10). Experiments conducted during development of embodiments of the present invention demonstrate that GM3 acts as a mediator in the impairment of diabetic wound healing, and that ganglioside depletion serves to reverse the wound healing defect in DIO diabetic mice.

### Example 2

### Means for In Vivo Ganglioside Depletion Through Topical Application

Two techniques were used to deplete gangliosides in in vitro studies and to accelerate wound healing in mouse models of diabetes: i) glucosylceramide synthase (GCS) inhibition with C9; and ii) gene suppression of GM3S. Newer small molecule inhibitors of GCS, such as C9 and EtDOP4, deplete GM3 (SEE FIG. 3E), including the increased KC GM3 with supplemental glucose (SEE FIG. 3F). In contrast to the first GCS inhibitor PDMP, neither C9 nor EtDOP4 increase ceramide (SEE FIGS. 1, 3G) (Lee et al. J Biol Chem 1999; 274:14662-9; Natoli et al. Nat Med 2010; 16:788-92; Wang et al. J Invest Dermatol 2006; 126:2687-96; Abe et al. J Lipid Res 1995; 36:611-21; herein incorporated by reference in their entireties). In fact, ceramide exacerbates obesity-associated insulin resistance (22, herein incorporated by reference in its entirety). GCS inhibitors show promise in reversing insulin resistance, hepatic steatosis and atherosclerosis associated with obesity (Zhao et al. Diabetes 2007; 56:1210-8; Aerts et al. Diabetes 2007; 56:1341-9; Yew et al. PLoS One 2010; 5:e11239; Zhao et al. Hepatology 2009; 50:85-93; Bietrix et al. Arterioscler Thromb Vasc Biol 2010; 30:931-7; herein incorporated by reference in their entireties). A related compound in phase II trials for Gaucher disease showed little toxicity (Lukina et al. Improvement in hematological, visceral, and skeletal manifestations of Gaucher disease type 1 with oral eliglustat tartrate (Genz-112638) treatment: two-year results of a Phase 2 study. Blood 2010; Lukina et al. Blood 2010; 116:893-9; herein incorporated by reference in their entireties). Prior studies have not addressed the cutaneous effects of these inhibitors.

### Example 3

### Gangliosides Impact on Keratinocyte Motility

Experiments conducted during development of the present invention indicate that GM3 mediates hyperglycemia- and cytokine-driven insulin resistance in diabetic skin. These findings were extended from observations of increased GM3 in diabetic mouse skin to evaluate the effect of genetic ganglioside depletion on wound healing in GM3 synthase knockout GM3S(GM3S^{-/-} or KO) mice. After 10 weeks on a high fat diet (HFD for DIO mice), GM3S^{-/-} KO mice (without GM3 in skin, SEE FIG. 2) were as obese as DIO littermates and as hyperglycemic (random blood glucose levels KO 187.7±43.5 mg/dl, WT 170.8±46.0 mg/dl); however, DIO KO mice have improved insulin sensitivity; at 120 mins after glucose challenge and overnight fast, DIO KO mouse glucose levels are 170.9±13.1, not different from regular diet (RD) WT mice (161.3±8.5 mg/dl), but much less than DIO WT mice (274.9±17.6 mg/dl) (n>12, each group) (Yamashita et al. Proc Natl Acad Sci USA 2003; 100:3445-9; herein incorporated by reference in its entirety). Using a splinted wound healing model that allows healing primarily by re-epithelialization and minimizes healing by contracture (Galiano et al. Wound Repair Regen 2004; 12:485-92; herein incorporated by reference in its entirety), marked acceleration in wound healing clinically (SEE FIG. 6A) and histologically (epidermal gap) (SEE FIG. 6B) was observed in these insulin-sensitive DIO KO mice in comparison with DIO WT mice. KO mice on a RD show normal laboratory testing, except for mild hypoglycemia with glucose tolerance testing (Yamashita et al. Proc Natl Acad Sci USA 2003; 100:3445-9; herein incorporated by reference in its entirety); their rate of wound healing is no different from RD GM3S^{+/+} mice. Interestingly, wound re-epithelialization in DIO KO mice at day 5 is even faster than in WT or KO RD mice (SEE FIG. 6B).

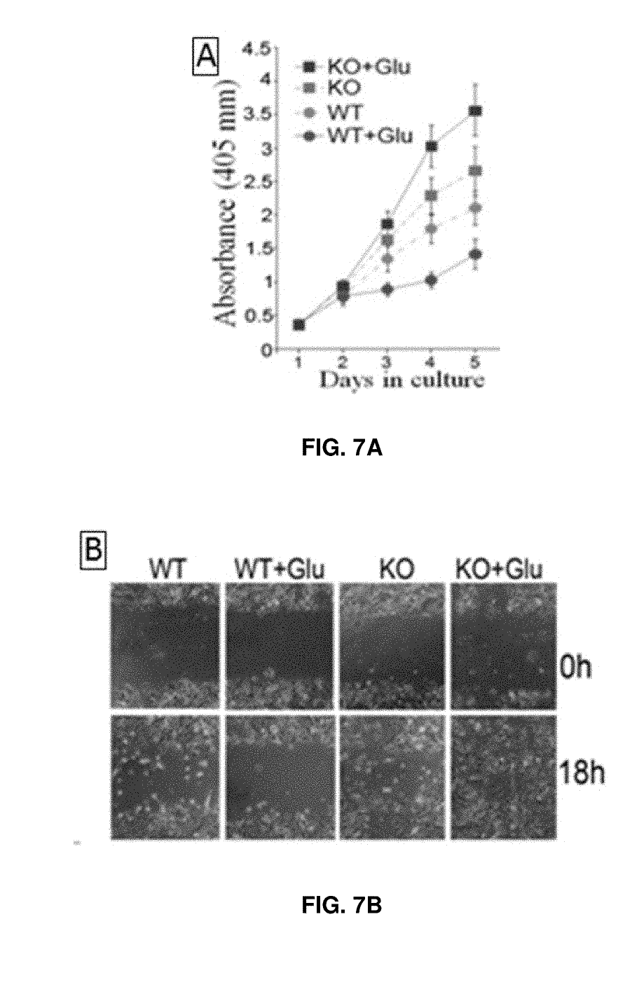

The accelerated wound healing in obese, hyperglycemic GM3S−/− mice indicates a role for gangliosides in regulating wound healing, but does not distinguish whether the impact of ganglioside depletion is by improving systemic insulin sensitivity or by a direct effect on KCs. It has been demonstrated that proliferation (SEE FIG. 7A) and migration (SEE FIG. 7B) of cultured WT KCs are suppressed by exposure to supplemental glucose, simulating hyperglycemia (Lan et al. Hyperglycaemic conditions decrease cultured keratinocyte mobility: implications for impaired wound healing in patients with diabetes. Br J Dermatol 2008; Spravchikov. Diabetes 2001; 50:1627-35; herein incorporated by reference in its entirety). This suppression is reversed in KO GM3S−/− mouse KCs. In fact, proliferation and migration are greater in the face of supplemental glucose in KO mice than without the glucose. The ability of cells to migrate efficiently across a wound depends on both maximal velocity and persistence of direction, which is strongly influenced by polarity. Ganglioside depletion in the KO mouse KCs accelerates the velocity of glucose-treated KCs (SEE FIG. 7B), but also increases cell persistence, regardless of whether in the presence of glucose (SEE FIG. 7C). These findings support a direct role of gangliosides on the proliferation and migration of KCs, particularly in the presence of increased glucose, which is known to suppress insulin signaling.

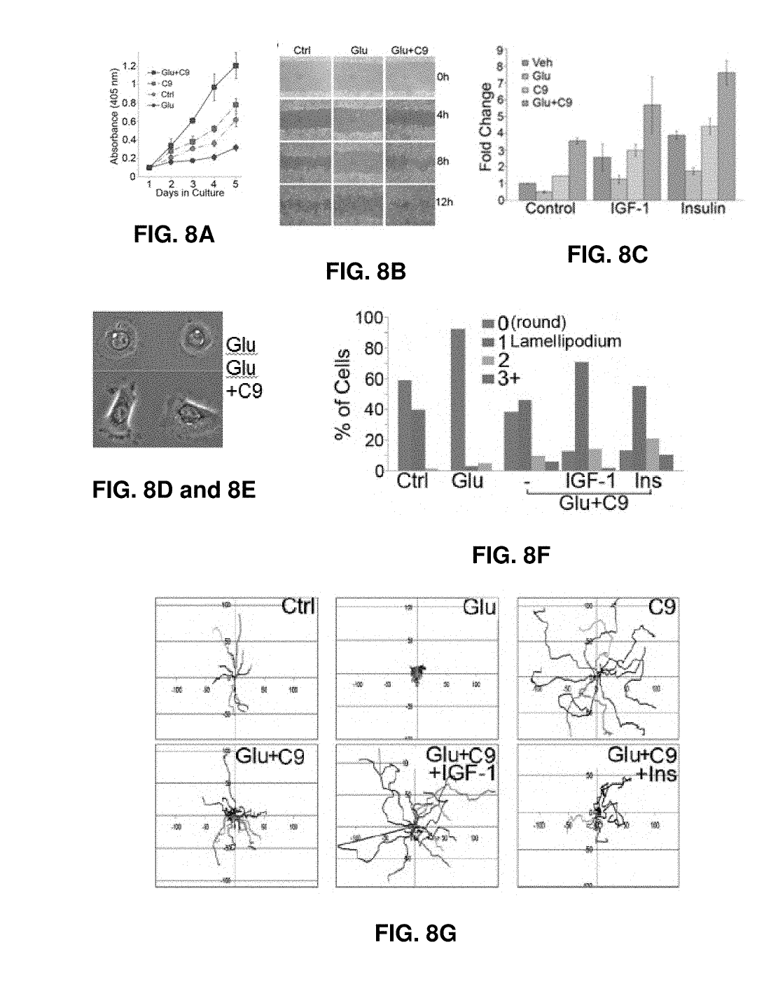

In human KCs, depletion of gangliosides by GCS inhibitors (C9 and EtDOP4) also increases cell proliferation (SEE FIG. 8A) and migration, particularly in response to insulin, IGF-1 (SEE FIG. 8B-E), and EGFR ligands (Wang et al. Cancer Res 2007; 67:9986-95; Wang et al. J Biol Chem 2002; 277:40410-9; Wang et al. J Biol Chem 2003; 278:25591-9; Wang et al. J Biol Chem 2003; 278:48770-8; herein incorporated by reference in their entireties). As suggested by the rapidity of KO mouse wound healing in vivo and seen in mouse KCs, proliferation and migration increase more dramatically in the presence of increased glucose. These data indicate that ganglioside depletion selectively improves wound healing in the presence of hyperglycemia. This selectivity is explained by the discovery that glucose exposure increases GM3 expression in HEKs (SEE FIG. 3C), implicating GM3 as a mediator of the insulin resistance from both hyperglycemia and chronic cytokine exposure and suggesting the unique utility of ganglioside depletion in healing wounds in a hyperglycemic milieu.

### Example 4

### Ganglioside Depletion Affects Rho GTPase Activity

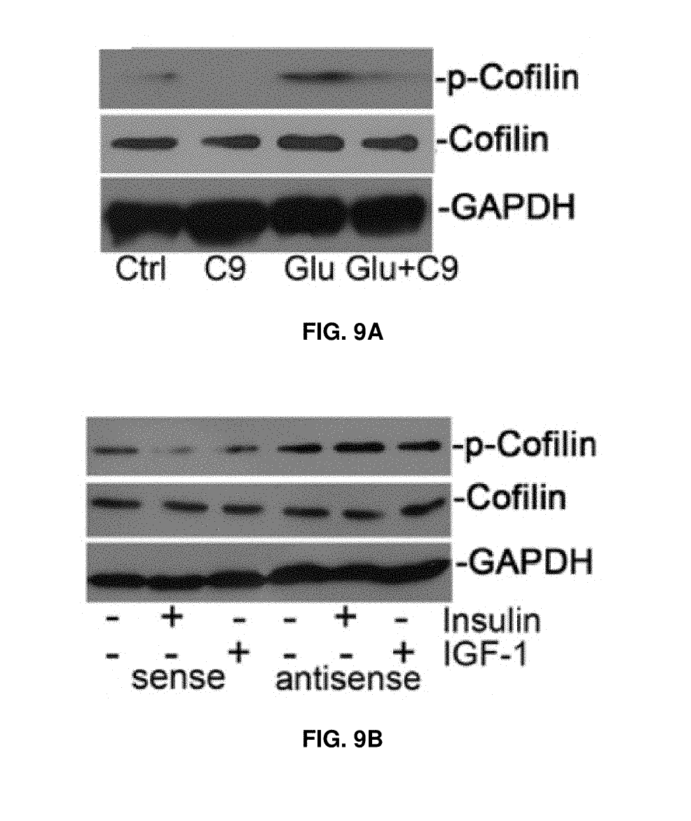

The alteration in cell shape and polarity when glucose-treated HEKs are “rescued” by C9 indicates that altered cytoskeletal organization contributes to the observed change in cell migration. Cofilin is a downstream effector of RhoA that reorganizes the actin cytoskeleton. Indeed, immunoblots show that phosphorylation of cofilin is increased by glucose and GD3S/GM2/GD2S antisense treatment (both associated with more GM3), whereas C9, insulin and IGF-1 decrease p-cofilin when GM3 is not increased (SEE FIG. 9). These data indicate that GM3 either suppresses RhoA activation or activates the cofilin phosphatase Slingshot 139-41. It is contemplated that GM3 expression increases the activity of Rac1 or possibly Cdc42, resulting in single lamellipodia formation and increased KC migration, although the present invention is not limited to any particular mechanism of action and an understanding of the mechanism of action is not necessary to practice the present invention. This is consistent with the demonstration that Rac1 activation is markedly suppressed in double knockout IR−/− IGF-1R−/− mouse KCs12.

### Example 5

### Diabetic Mouse Skin Shows Increased GM3 Synthase and Ganglioside GM3

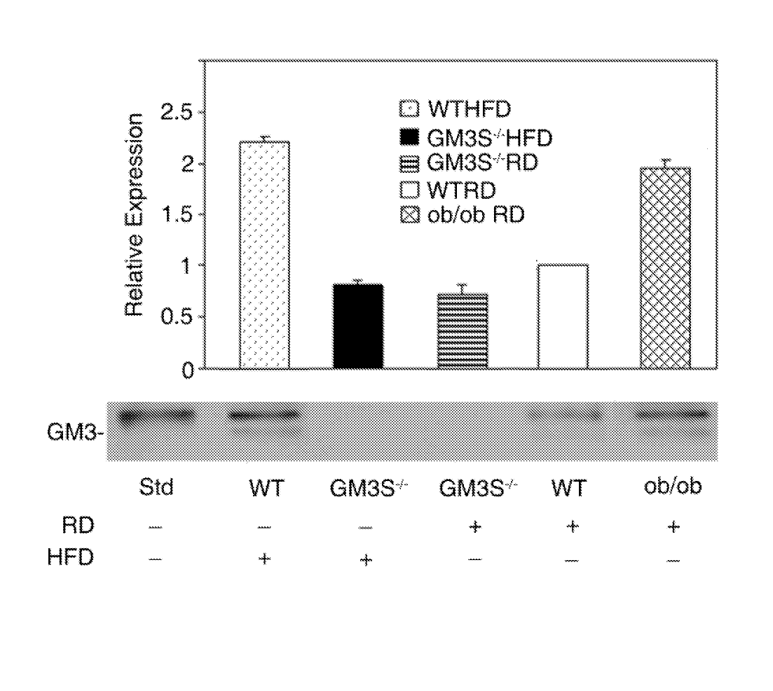

Experiments were conducted during development of embodiments of the present invention to determine if skin in diabetic mice accumulates GM3, as has been shown in adipose tissue and muscle. Epidermis and dermis were separated from the underlying adipose tissue in two diabetic mouse models: ob/ob and a model that more closely simulates type 2 diabetes, diet-induced obese (DIO) C57BL/6 mice fed a high-fat diet (HFD) for 10 weeks. In comparison with levels in C57BL/6 mice fed a regular diet (RD), diabetic skin showed a 2-fold increase in the expression of GM3S, as demonstrated by qRT-PCR (FIG. 11A), and a 4-fold increase in ganglioside GM3, as shown by thin layer chromatography (TLC) immunostaining (FIG. 11B).

### Example 6

### GM3S Depletion Reverses the Wound Healing Defect in Diet-Induced Obese Mice

Experiments were conducted during development of embodiments of the present invention to determine if the increase in GM3 and GM3S expression contributed to the delayed wound healing of diabetes. Wound healing was compared between GM3S^{-/-} and WT littermate mice, including mice administered a 10 wk HFD to induce obesity and diet-induced type 2 diabetes. Yamashita et al. have previously shown improved responses to insulin and glucose challenge in GM3S^{-/-} mice vs. WT littermates (Yamashita et al. 2003. Proc Natl Acad Sci USA 100:3445-3449, herein incorporated by reference in its entirety), but responses in skin tissue have not been examined. Experiments verified the metabolic characteristics of GM3S^{-/-} mice on a RD and 10 wks on a HFD in comparison with WT littermates (Table 1).

TABLE 1
Metabolic characterization of GM3S^{-/-} mice

| | | Fasting | | | | Fed |
|---|---|---|---|---|---|---|
| | Weight | glucose | 30 min GTT | 60 min GTT | 120 min GTT | insulin |
| Mouse set | (g) | (mg/dl) | (mg/dl) | (mg/dl) | (mg/dl) | (ng/ml) |
| WT HFD | 32.2 ± 0.5 | 112.2 ± 4.7 | 465.1 ± 13.9 | 444.9 ± 18.0 | 264.7 ± 14.0 | 1.8 ± 0.9 |
| | n = 40 | | | | | |
| KO HFD | 30.8 ± 0.7 | 97.5 ± 5.8 | 420.8 ± 12.8 | 326.5 ± 19.2 | 169.9 ± 10.8 | 0.7 ± 0.5 |
| | n = 27 | | | | | |
| WT RD | 25.8 ± 0.4 | 89.3 ± 4.4 | 217.0 ± 17.9 | 188.5 ± 4.4 | 161.3 ± 8.5 | 0.7 ± 0.4 |
| | n = 27 | | | | | |
| KO RD | 26.9 ± 0.4 | 94.4 ± 6.0 | 332.6 ± 34.0 | 193.0 ± 13.6 | 119.0 ± 5.2 | 0.8 ± 0.5 |
| | n = 17 | | | | | |
| p-values: | | | | | | |
| KO HFD vs WT HFD | 0.15 | 0.10 | 0.006 | <0.001 | <0.001 | 0.003 |
| WT HFD vs WT RD | <0.001 | 0.003 | <0.001 | <0.001 | <0.001 | 0.002 |
| KO HFD vs KO RD | <0.001 | 0.51 | 0.06 | <0.001 | <0.001 | — |
| KO HFD vs WT RD | <0.001 | 0.23 | <0.001 | <0.001 | 0.54 | 0.994 |
| KO RD vs WT RD | 0.09 | 0.51 | 0.007 | 0.003 | 0.002 | 0.987 |

GTT = glucose tolerance testing;
ND = not done

WT HFD mice showed higher weights, glucose levels in glucose tolerance testing (at all times tested), and fasting glucose levels than WT RD mice. Although GM3S^{-/-} HFD mice were as obese as their WT HFD littermates, their responses to glucose challenge and fed insulin levels were improved (Table 1). Although 30 and 60 min glucose levels were high, by 120 min after glucose challenge, serum glucose levels in GM3S^{-/-} HFD mice were no different from levels in WT RD mice, and were 64% of levels in WT HFD mice. GM3S^{-/-} RD mice became hypoglycemic 120 min after glucose challenge. GM3S^{-/-} mice showed no detectable GM3 by TLC immunostaining or GM3S mRNA expression, regardless of diet (FIG. 11).

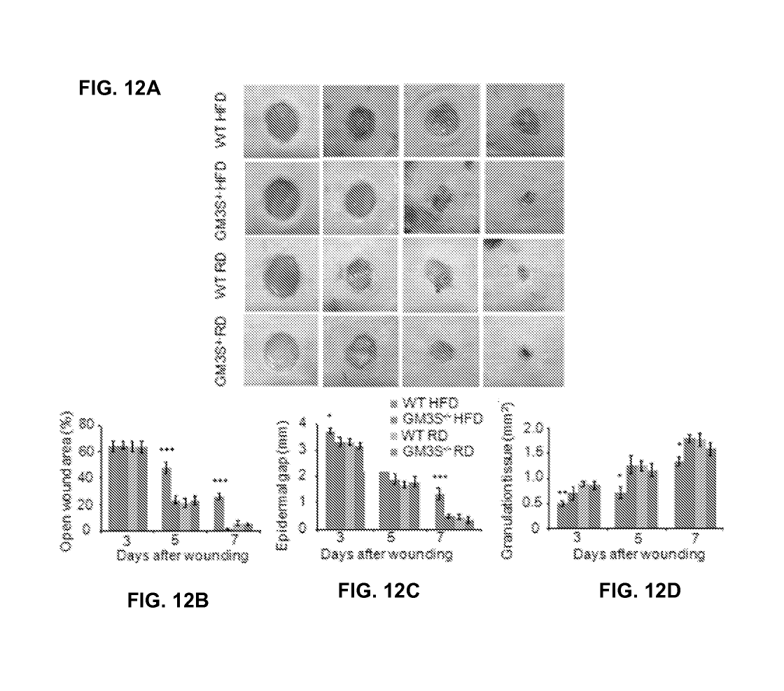

To compare wound healing in GM3S^{-/-} mice and their WT littermates, a silicone splinted wound healing model was used that allows healing primarily by re-epithelialization and minimizes healing by contracture (Galiano et al. 2004. Wound Repair Regen 12:485-492; herein incorporated by reference in its entirety), thus more closely approximating human wound healing. Wound closure was visibly delayed in WT HFD mice beginning 5 days after wound initiation (FIGS. 12A, 12B), but wound closure in the GM3S^{-/-} HFD mice was no different from that of mice on a RD. By 7 days after wounding, wound areas were 17-fold larger in WT HFD mice than in GM3S^{-/-} HFD mice. The epidermal gap as a histological measure of epidermal closure (FIG. 12C) was first noted to be different in WT HFD vs. other mouse groups, including GM3S^{-/-} HFD mice, at 3 days after wound initiation, and by 5 and 7 days after wounding, the difference was progressively greater; by 7 days after wounding the gap in WT HFD mouse wounds was 3-fold larger than that of GM3S^{-/-} HFD mice. Epidermal closure required a mean of 14.1 days for wounds to fully close in the WT HFD mice, but 7.4 days in GM3S^{-/-} HFD mice. Epidermal closure histology was not different between GM3S^{-/-} HFD mice and mice on a RD at any time point. The area of granulation tissue was significantly smaller in WT HFD wounds than in wounds from either WT RD or GM3S^{-/-} HFD mice (FIG. 12D). The area of granulation tissue did not differ between WT RD and GM3S^{-/-} HFD mouse wounds at any time point.

To address the potential impact of increased KC migration and proliferation in the accelerated re-epithelialization in GM3S^{-/-} mice, proliferating KCs were labeled with BrdU. The extent of migration, as determined by the length of basal epidermis from the wound edge to the first proliferating KC, was significantly less in WT HFD epidermis (129.9±34.8 m) than in WT RD (193.5±42.7 m) and GM3S^{-/-} HFD (216.3±41.5 m) epidermis. The GM3S^{-/-} HFD epidermis migrational distance did not differ from that of RD epidermis. The percentage of proliferating basal KCs in WT HFD epidermis (34.8±2.4) was significantly lower than in either GM3S^{-/-} HFD epidermis (41.5±1.7) or WT RD epidermis (42.7±1.7). Basal keratinocyte proliferation was similar in GM3S^{-/-} HFD and WT RD epidermis.

### Example 7

### Supplemental Glucose Increases KC Expression of GM3 and GM3S In Vitro, and Suppresses KC Migration and Proliferation

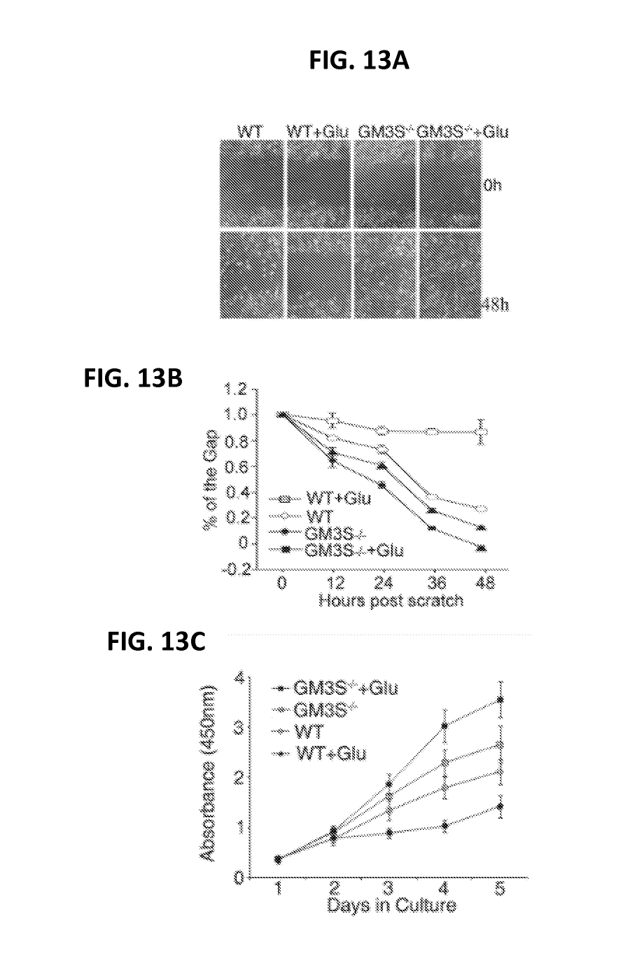

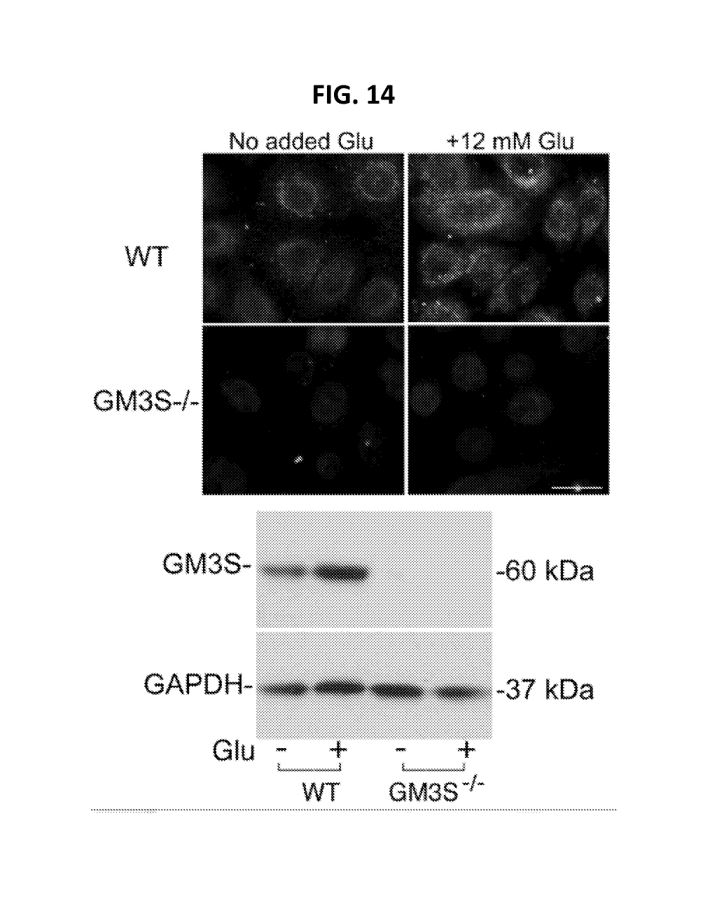

Increased glucose in medium has traditionally been used as a surrogate for the hyperglycemic diabetic environment Suzuki et al. 2011. Proc Natl Acad Sci USA 108:13829-13834; Ingram et al. 2008. Diabetes 57:724-731; herein incorporated by reference in their entireties). Pre-incubation of WT primary mouse KCs in the presence of 12 mM supplemental glucose for 72 h virtually ceased migration in scratch assays (“in vitro wounds”) performed in the presence of mitomycin C to prevent proliferation (FIGS. 13A, B), and markedly reduced cell proliferation in WST assays (p<0.001 by 48 h) (FIG. 13C). Treatment with glucose-supplemented medium increased GM3 expression, as seen by confocal immunofluorescence microscopy (FIG. 14A, top) and doubled GM3S expression by Western immunoassays (FIG. 14B, 2^{nd} VS. 1^{st} lane).

### Example 8

### GM3 Depletion Accelerates Mouse KC Migration and Proliferation, Especially in the Presence of Increased Glucose

Experiments were conducted during development of embodiments of the present invention to test the direct effect of GM3S depletion on mouse KC migration and proliferation in vitro. In scratch assays GM3S^{-/-} cells migrated faster in normoglycemic, serum-containing medium than WT cells by 24 h after the scratch, and were resistant to the inhibitory effects of glucose. In hyperglycemic medium GM3S^{-/-} KCs paradoxically closed the scratch faster than GM3S^{-/-} or WT cells without supplemental glucose (FIGS. 13A, B). Similarly, hyperglycemic medium suppressed proliferation of WT KCs, but dramatically stimulated proliferation of GM3S^{-/-} KCs (FIG. 13C). Immunofluorescence staining and Western blotting showed no detectable GM3 and GM3S expression, respectively (FIG. 14A, bottom and FIG. 14B, right).

GM3S knockout reduces GM3 expression, but also leads to accumulation of the GM3S substrate, lactosylceramide (LacCer) (Hashiramoto et al. 2006. Oncogene 25:3948-3955; herein incorporated by reference in its entirety). In contrast to the observed increase in migration and proliferation of GM3S^{-/-} KCs, WT KCs treated with supplemental LacCer showed slightly decreased cell migration or proliferation, regardless of glucose concentration. This result further implicates GM3 depletion as the key driver of accelerated KC re-epithelialization.

### Example 9

### GM3 Depletion Prevents Glucose-Induced Suppression of Single-Cell Directional Movement

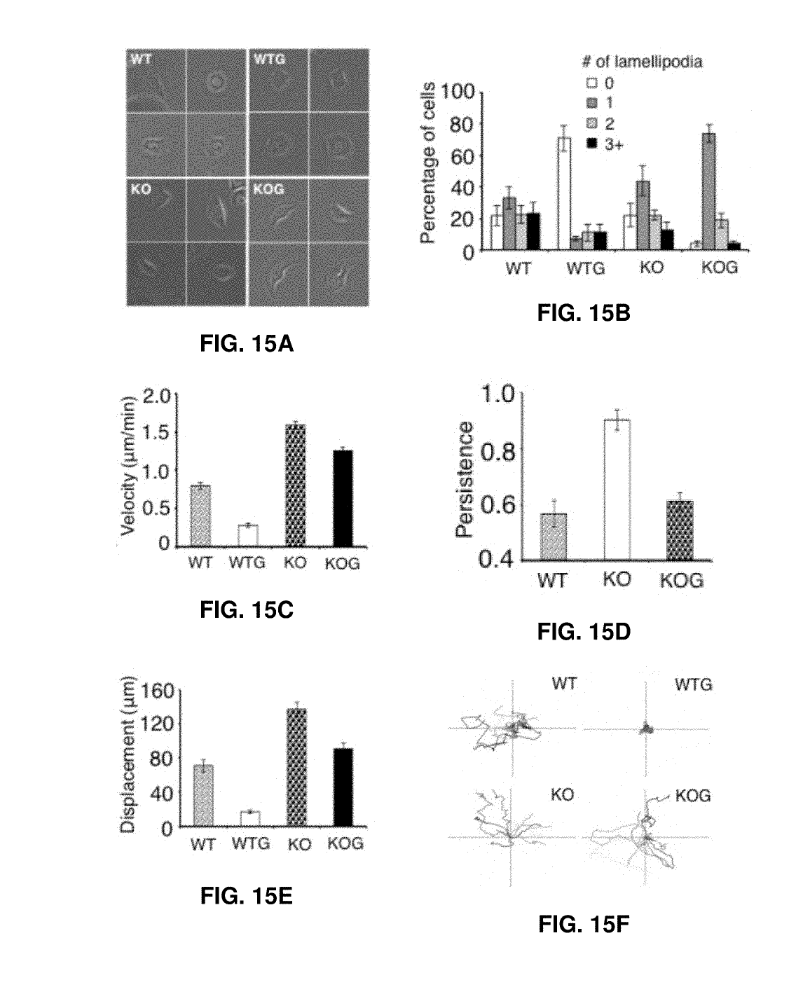

Live cell imaging studies showed that increased glucose exposure altered the morphology of WT KCs, causing them to round in shape with loss of lamellipodia (FIG. 15A, upper right, and 15B). In contrast, GM3S^{-/-} KCs not only retained lamellipodia, but also more often showed a single lamellipodium despite exposure to glucose (FIG. 15B). A single lamellipodium (FIG. 15, lower right) was noted in 73.6% of GM3S^{-/-} KCs grown in hyperglycemic medium, but in only 6.9% of WT KCs grown in hyperglycemic medium; the majority of these single lamellipodia in GM3S^{-/-} KCs were noted to be large and sail-shaped (FIG. 15A). Single cell live imaging studies showed that the velocity of GM3S^{-/-} KCs was higher than WT cells and, in contrast to WT cells, was not reduced by supplemental glucose (FIG. 15C). Persistence, which measures how much cells migrate in a given direction, was higher in GM3S^{-/-} KCs than in WT KCs (FIG. 15D). In the presence of glucose, WT cells barely moved at all (i.e., persistence not measurable). Persistence of GM3S^{-/-} KCs cells in hyperglycemic medium resembled that of WT KC in normoglycemic medium. The result of the increases in both velocity and persistence was a net increase in displacement; exposure to supplemental glucose reduced net displacement of WT KCs by 85%, but GM3S^{-/-} KCs treated with glucose showed a net displacement that was still 25% higher than that of WT KCs in standard medium and 6-fold that of WT cells exposed to glucose (FIG. 15E). Vector maps similarly showed virtually no movement in WT KCs in hyperglycemic medium, but vigorous movement and displacement in GM3S^{-/-} KC, even in the presence of supplemental glucose for more than 72 h (FIG. 15F).

### Example 10

### GM3S Depletion Activates IR and IGF-1R Signaling, Particularly in the Presence of Supplemental Glucose

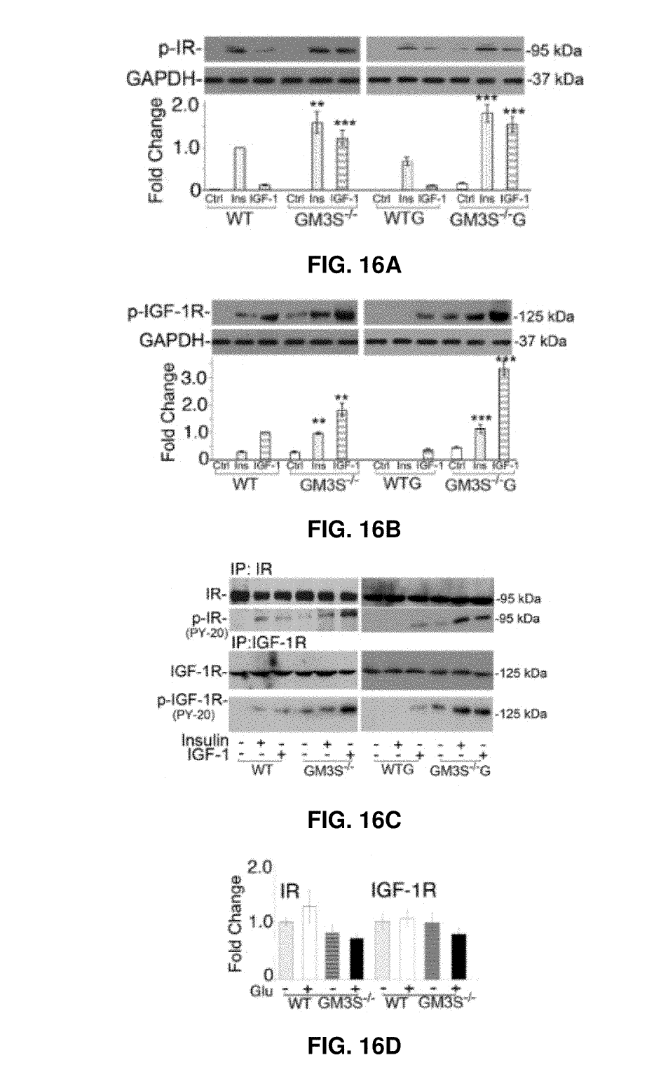

When cells were grown in normoglycemic medium before overnight starvation and insulin stimulation, phosphorylation of IR, as detected directly with p-IR antibody, was 1.4-fold greater in GM3S^{-/-} KCs than in WT KCs (FIG. 16A, top row, lane 2 vs. 5). Under similar conditions, IGF-1 induced 7-fold greater p-IR in GM3S^{-/-} KCs than in WT KCs (FIG. 16A, top row, lane 3 vs. 6). When WT cells were exposed to hyperglycemic medium for 72 h, p-IR responses to insulin and IGF-1 were reduced by 38% and 16%, respectively, but hyperglycemic medium increased the p-IR responses to insulin and IGF-1 in GM3S^{-/-} KCs by 37% and 22%, respectively (FIG. 16A, lanes 7-12).

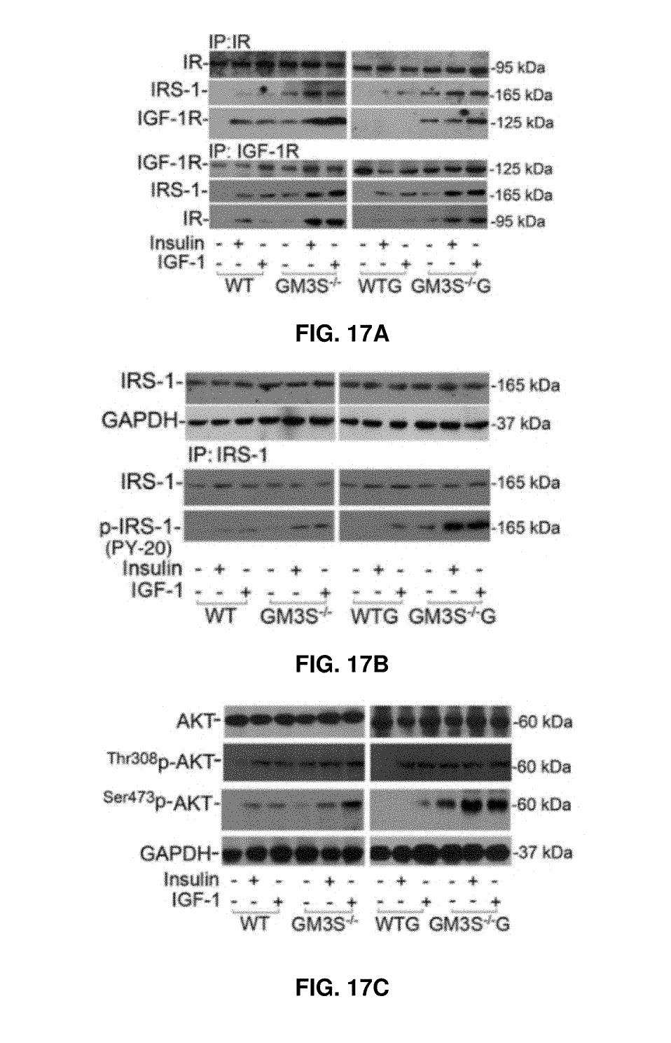

In normoglycemic medium, GM3S deficiency increased phosphorylation of IGF-1R, as detected by p-IGF-1R antibody, by 1.9-fold after IGF-1 stimulation and by 2.5-fold after insulin stimulation (FIG. 16B, top row, lane 2 vs. 5 and 3 vs. 6). In hyperglycemic medium, p-IGF-1R after exposure to either insulin or IGF-1 was markedly reduced in WT KCs, but in GM3S^{-/-} KCs insulin-stimuated p-IGF-1R was maintained and IGF-1-stimulated p-IGF-1 was increased (FIG. 16B, top row). Even in the absence of growth factor stimulation, IGF-1R was activated in GM3S^{-/-} primary KCs, and this phosphorylation was increased by 37% in the presence of supplemental glucose (FIG. 17B, top row, lanes 4 and 10).

IR and IGF-1R were each immunoprecipitated and phosphorylation was detected with PY-20 antibody. As with direct detection of phosphorylated receptors, GM3S knockout increased the phosphorylation of IR and IGF-1R, including in the absence of growth factor exposure (FIG. 16C). While IR and IGF-1R phosphorylation was suppressed by exposure to increased glucose in WT KCs (lanes 2-3 vs. 8-9), receptor phosphorylation was maintained and even increased in GM3S^{-/-} KCs (lanes 4-6 vs. 10-12). Neither GM3S depletion nor exposure to glucose significantly altered the expression of IR or IGF-1R mRNA (FIG. 16D).

### Example 11

### GM3S Depletion Triggers Activation of IRS-1 and Downstream Signaling

Activation of IR or IGF-1R leads to formation of a complex with insulin receptor substrates (IRS). In KCs, insulin pathway signaling activates phosphatidylinositol 3-kinase (PI3K) via IRS-1 (and not IRS-2) (Sadagurski et al. 2005. J Biol Chem 280:14536-14544; herein incorporated by reference in its entirety), indicating that the association of IR and IGF-1R with IRS-1 is key for downstream signaling transduction, and tyrosine phosphorylation of IRS-1. Stimulation of WT KCs with insulin or IGF-1 led to the association of IRS-1 with immunoprecipitated IR (FIG. 17A, 3rd row, lanes 2 and 3, respectively) and immunoprecipitated IGF-1R (FIG. 17A, 5th row, lanes 2 and 3, respectively). In the GM3S^{-/-} KCs, the growth factor induced association of IRS-1 with IR (FIG. 17A, 3rd row, lanes 5 and 6) and with IGF-1R (FIG. 17A, lanes 5 and 6) was much stronger than that seen in WT cells, and the association in GM3S^{-/-} KCs was even greater without growth factor stimulation (lane 4) than in WT cells with growth factor. In stimulated WT cells, IGF-1R was also associated with IR and vice versa, and this association was greatly increased by GM3S depletion (FIG. 17A, rows 3 and 6). Interestingly, glucose supplementation had only a small effect on IRS-1 association with each receptor in WT cells, but virtually eliminated the IR-IGF-1R association; in GM3S^{-/-} cells, the IR-IGF-1R association was reduced, but maintained. IRS-1 phosphorylation, as detected by IRS-1 immunoprecipitation and PY-20 antibody, was also increased by GM3S depletion, particularly in the face of glucose excess (FIG. 17B, bottom row). IRS-1 expression was not altered by GM3S depletion or glucose exposure (FIG. 17B, top row). Similarly, GM3S depletion did not affect AKT expression (FIG. 17C, top row), but markedly increased insulin- and IGF-1 induced phosphorylation of p-AKT at Ser 473, including without growth factor stimulation and most potently in the presence of increased glucose (FIG. 17C, 3rd row).

### Example 12

### Materials and Methods

### Mouse Models and Cultured Mouse Keratinocytes.

Mouse studies were approved by the Northwestern Animal Care and Use Committee. GM3 synthase knockout (GM3S^{-/-)} mice (Yamashita et al. 2003. Proc Natl Acad Sci USA 100:3445-3449; herein incorporated by reference in its entirety) were backcrossed 6 times to C57BL/6 mice (Jackson Laboratory) to produce fully congenic GM3S^{-/-} mice and their wildtype littermate controls (GM3S^{+/+}, WT). Male mice were fed either a regular diet (RD) containing 11.4% fat, 62.8% carbohydrate, and 25.8% protein (total 12.6 kJ/g, Harlan Teklad 7012) or a high fat diet (HFD) consisting of 42% fat, 25.6% carbohydrate, and 16.4% protein (total 23.4 kJ/g, Harlan Teklad TD88137) for 10 weeks and throughout analyses to establish and maintain diet-induced obesity. For in vitro studies, mouse KCs were isolated from GM3S^{-/-} or WT mouse skin at 1 day of age. After washing, mouse skin was incubated overnight at 4° C. in complete CnT-07 medium (ZenBio, Research Triangle Park, N.C.) with dispase II (5 mg/mL, Roche, Indianapolis, Ind.) to separate epidermis from dermis. Epidermis was then incubated with TrypLE Select (Invitrogen, Carlsbad, Calif.) for 30 min at room temperature and individual KCs further dispersed by pipetting before plating in CnT-07 complete medium (ZenBio, Research Triangle Park, N.C.). Primary cultured mouse KCs were used at passages 2-3 for all studies.

### Detection and Measurement of Ganglioside GM3.

Total ganglioside was extracted as previously described (Wang et al. 2001. J Invest Dermatol 116:69-76; herein incorporated by reference in its entirety) from mouse skin (ob/ob; GM3S^{-/-} and WT mice on either a regular or high fat diet for 10 wks). TLC immunostaining using anti-GM3 antibody (DH2, Glycotech, MD) was performed to determine GM3 ganglioside expression (Wang et al. 2002. J Biol Chem 277:47028-47034; herein incorporated by reference in its entirety). Ganglioside GM3 was also detected in mouse KCs with and without supplementation with 12 mM glucose by immunofluorescence using anti-GM3 antibody (Seikagaku Corp. Japan). For immunofluorescence, primary cultured mouse KCs at 80% confluence on glass cover slips were fixed in 4% methanol-free paraformaldehyde for 10 min at 4° C. before incubating with 2% BSA-PBS to block non-specific binding for 1 h at RT. After incubation with anti-GM3 antibody overnight at 4° C. followed by washing with 1% BSA-PBS for 30 min at RT, FITC-labeled goat anti-mouse antibody was incubated with cells for 45 min at RT and counterstained with 10 mM L-4′,6-diamidino-2-phenylindole (DAPI). Images were captured using the UV LSM 510 Meta confocal imaging system (Zeiss, MA).

Quantification of mRNA Expression.

qRT-PCR was performed using cDNA made from total RNA isolated from mouse skin and mouse KCs using the 7000 Sequence Detection System (ABI PRISM). Trizol reagent was used to extract total RNA (Invitrogen, CA) and cDNA was synthesized using qScript™ cDNA SuperMix (Quanta Biosciences, MD) following manufacturer instructions.

### Glucose Tolerance Tests and Insulin Measurement.

For glucose tolerance testing (GTT), mice were fasted overnight before injecting i.p. with D-glucose (2 g/kg). Blood was obtained by tail nick at baseline, 30, 60, and 120 min after glucose administration (Yamashita et al. 2003. Proc Natl Acad Sci USA 100:3445-3449; herein incorporated by reference in its entirety). For measuring insulin, whole blood obtained from the intraorbital retrobulbar plexus was allowed to clot at RT for 30 min before centrifugation at 4° C. to separate serum from clotted blood component, and serum was stored at −80° C. until insulin was measured using Ultra Sensitive Mouse Insulin ELISA Kit (Crystal Chem INC. 90080). Absorbance was read at 450 nm in a plate reader (EL808 microplate reader, BIO-TEK, Inc) linked with the KC Junior program.

### In Vivo Wound Healing Analysis Using Splints to Prevent Contraction.

The dorsal surface of mice was shaved, depilated, and wounded 24 h later (20). 5 mm wounds were made with a punch biopsy on each side of the midline to below the level of the panniculus carnosus. A donut-shaped silicone splint with a 10 mm outer diameter and a 6 mm inner diameter was centered in the wound and fixed to the skin using an immediate-bonding adhesive (Krazy Glue, Elmer's Inc., Columbus, Ohio) and interrupted 6-0 nylon sutures (Ethicon Inc., Somerville, N.J.). A semiocclusive dressing (Tegaderm, 3M, St. Paul, Minn.) was then applied to the wound over a nonstick (Telfa) pad. The wound area was analyzed at day 3, 5 and 7 post wounding (and every 3 days until fully healed visually) by tracing the wound margin and calculating the pixel area using digital imaging (Axiovision version 4.5) of photographs taken at 5 cm from the mouse. The wound area was calculated as a percent area of the original wound area. Because the splint has a constant area, it served as a visual cue to the initial size of the wound. Wounds were harvested on days 3, 5, and 7 with a 10 mm punch biopsy centered in the wound, and processed for histologic and RNA expression studies.

### Histological Studies.

All histological and immunohistochemical studies were performed in the Northwestern University Skin Disease Research Center's Pathology Core facility. Harvested wounds were paraffin-embedded for routine histological and immunohistochemical analyses (45, 46). Stained 4 μm sections were photographed at 10× magnification under light microscopy (Zeiss Axioplan 2 imaging, Thornwood N.Y.), and imaged digitally (Axiovision version 4.5) to measure the epidermal gap and area of granulation tissue. The epidermal gap was defined as the maximal gap between the leading edges of epidermal migration, with an epidermal gap of 0 μm representing a completely reepithelialized wound. The area of granulation tissue was calculated by tracing regions of granulation tissue, calculating pixel area, and adding the areas of these regions. At least 28 wound specimens per group were analyzed at each time point. For BrdU incorporation testing in 3-day wounds, mice were injected intraperitoneally with BrdU (30 μg/g) 2 h before sacrifice. Labeling was detected immunohistochemically with anti-BrdU antibody (Developmental Studies Hybridoma Bank, Iowa City, Iowa). Using AxioVision computer-assisted morphometric software (Carl Zeiss), the percentage of basal KCs with BrdU-labeled nuclei was determined in the proliferating segment, as defined by the first labeled KC proximal to the wound margin to a labeling index of less than 10% of KCs. KC migration across the wound was quantified as the length of basal epidermis from the wound edge to the first BrdU-labeled KC. Cells were counted by two pre-trained blinded observers. Sections from at least 28 wounds per group were assessed in each set for BrdU staining.

### In Vitro Proliferation, Migration and Motility Assays.

To simulate chronic hyperglycemia with a 2-3-fold increase in glucose concentration, mouse KC medium (with 8 mM glucose) was supplemented with 12 mM glucose (Sigma, St. Louis, Mo.) for 72 h. For proliferation assays, KCs were plated onto 96-well (2×10³ cell/well) or 12-well (2×10⁴ cell/well) plates and cell proliferation was assessed daily for 5 d by either WST assay (96-well plate) per manufacturer's instruction (Clontech, CA) and manual counting using a hemacytometer (12-well plate). Cell migration was assessed by in vitro wound (scratch) assays as previously described (47). In brief, confluent monolayer KCs were treated with 5 μg/mL mitomycin for 1 h to prevent proliferation before the scratch was made with a 10 μL pipette tip. Cell migration in complete CnT-07 medium in the presence of 4 μg/mL mitomycin was recorded photographically every 3-4 h for 60 h under an inverted phase-contrast microscope (Nikon). The unfilled scratch area was measured using AxioVision software and recorded as the total pixels of the open area. The ratio of total pixels of unfilled area/the total pixels of initial unfilled (scratched) area among various groups were compared at each time point. Proliferation and migration studies were also performed in WT KCs with supplemental, given the known accumulation of LacCer, in addition to GM3 depletion, in GM3S^{-/-} mouse cells, in GM3S^{-/-} and WT cells with the PI3K inhibitor LY290004.

To analyze the effect of GM3S knockout on specific migration characteristics, KCs were plated onto collagen I-coated glass plates and allowed to attach for 2 h. Single live cell motility was monitored using the Nikon BioStation for 2 h, with images taken every 2 mins. Images were analyzed using ImageJ software. Lamellipodia were quantified by scoring the number of sheet-like extensions in phase-contrast images of live cells as described. Velocity, persistence, and final displacement from the origin were measured for each cell using the Manual Tracking plug-in. Plane-coordinates from the Manual Tracking plug-in were used to generate vector maps using Microsoft Excel. Micrometer units were calibrated using the scale bar provided on the Biostation platform. At least 100 single KCs were analyzed per group.

### Immunoblotting.

Primary mouse KCs were treated with or without 12 mM supplemental glucose in complete medium for 72 h. Cells were then starved of growth supplements, and stimulated with or without insulin (5 μg/mL, Sigma) for 15 min or IGF-1 (100 ng/mL, Prospect, East Brunswick, N.J.) for 30 mins. These selected times represented maximal stimulation times for each growth factor based on preliminary studies with exposure times of 5, 15, 30 and 60 mins, each was performed 3-5 times. Total protein from whole cell lysate was harvested in RIPA buffer. Monoclonal antibody directed against p-IR was from Millipore (MA), secondary antibodies were from Jackson ImmunoResearch Labs (PA), anti-IR, anti-IGF1-R, anti-p-IGF1-R, anti-GM3S, and anti-IRS-1 were polyclonal antibodies from Santa Cruz (Calif.), anti-IRS-1 monoclonal antibody, PY-20, anti-AKT, anti-AKT-serine473, and anti-AKT-threonine308 were from Cell Signaling Technology (MA). GAPDH expression was probed with anti-GAPDH antibody (Santa Cruz, Calif.) as a loading control. Band density was assessed using the ImageJ program and differences in receptor phosphorylation among groups were compared after normalization based on GAPDH band density.

Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### REFERENCES

All publications and patents cited in the present application and/or listed below are herein incorporated by reference in their entireties.
Lee L, Abe A, Shayman J A. Improved inhibitors of glucosylceramide synthase. J Biol Chem 1999; 274:14662-9.
Natoli T A, Smith L A, Rogers K A, et al. Inhibition of glucosylceramide accumulation results in effective blockade of polycystic kidney disease in mouse models. Nat Med 2010; 16:788-92.
Wang X Q, Sun P, Paller A S. Ganglioside induces caveolin-1 redistribution and interaction with the epidermal growth factor receptor. J Biol Chem 2002; 277:47028-34.
Wang X, Sun P, Al-Qamari A, Tai T, Kawashima I, Paller A S. Carbohydrate-carbohydrate binding of ganglioside to integrin alpha(5) modulates alpha(5)beta(1) function. J Biol Chem 2001; 276:8436-44.
CDC. National diabetes fact sheet: general information and national estimates on diabetes in the United States, 2005. Atlanta (Ga.): US Department of Health and Human Services, Centers for Disease Control and Prevention http://wwwcdcgov/diabetes/pubs/factsheet07htm 2007.
Hotamisligil G S, Murray D L, Choy L N, Spiegelman B M. Tumor necrosis factor alpha inhibits signaling from the insulin receptor. Proc Natl Acad Sci USA 1994; 91:4854-8.
Goren I, Muller E, Pfeilschifter J, Frank S. Severely impaired insulin signaling in chronic wounds of diabetic ob/ob mice: a potential role of tumor necrosis factor-alpha. Am J Pathol 2006; 168:765-77.
Yu J, Peng H, Ruan Q, Fatima A, Getsios S, Lavker R M. MicroRNA-205 promotes keratinocyte migration via the lipid phosphatase SHIP2. Faseb J 2010; 24:3950-9. Galiano R D, Michaels J, Dobryansky M, Levine J P, Gurtner G C. Quantitative and reproducible murine model of excisional wound healing. Wound Repair Regen 2004; 12:485-92.
Li Y, Fan J, Chen M, Li W, Woodley D T. Transforming growth factor-alpha: a major human serum factor that promotes human keratinocyte migration. J Invest Dermatol 2006; 126:2096-105.
Ando Y, Jensen P J. Epidermal growth factor and insulin-like growth factor I enhance keratinocyte migration. J Invest Dermatol 1993; 100:633-9.
Stachelscheid H, Ibrahim H, Koch L, et al. Epidermal insulin/IGF-1 signalling control interfollicular morphogenesis and proliferative potential through Rac activation. Embo J 2008; 27:2091-101.
Haase I, Evans R, Pofahl R, Watt F M. Regulation of keratinocyte shape, migration and wound epithelialization by IGF-1- and EGF-dependent signalling pathways. J Cell Sci 2003; 116:3227-38.
Tagami S, Inokuchi Ji J, Kabayama K, et al. Ganglioside GM3 participates in the pathological conditions of insulin resistance. J Biol Chem 2002; 277:3085-92.
Memon R A, Holleran W M, Uchida Y, et al. Regulation of glycosphingolipid metabolism in liver during the acute phase response. J Biol Chem 1999; 274:19707-13.
Yamashita T, Hashiramoto A, Haluzik M, et al. Enhanced insulin sensitivity in mice lacking ganglioside GM3. Proc Natl Acad Sci USA 2003; 100:3445-9.
Zhao H, Przybylska M, Wu I H, et al. Inhibiting glycosphingolipid synthesis improves glycemic control and insulin sensitivity in animal models of type 2 diabetes. Diabetes 2007; 56:1210-8.
Aerts J M, Ottenhoff R, Powlson A S, et al. Pharmacological inhibition of glucosylceramide synthase enhances insulin sensitivity. Diabetes 2007; 56:1341-9.
Yew N S, Zhao H, Hong E G, et al. Increased hepatic insulin action in diet-induced obese mice following inhibition of glucosylceramide synthase. PLoS One 2010; 5:e11239.
Wang X Q, Sun P, Go L, Koti V, Fliman M, Paller A S. Ganglioside GM3 promotes carcinoma cell proliferation via urokinase plasminogen activator-induced extracellular signal-regulated kinase-independent p70S6 kinase signaling. J Invest Dermatol 2006; 126:2687-96.
Abe A, Radin N S, Shayman J A, et al. Structural and stereochemical studies of potent inhibitors of glucosylceramide synthase and tumor cell growth. J Lipid Res 1995; 36:611-21.
Holland W L, Brozinick J T, Wang L P, et al. Inhibition of ceramide synthesis ameliorates glucocorticoid-, saturated-fat-, and obesity-induced insulin resistance. Cell Metab 2007; 5:167-79.
Zhao H, Przybylska M, Wu I H, et al. Inhibiting glycosphingolipid synthesis ameliorates hepatic steatosis in obese mice. Hepatology 2009; 50:85-93.
Bietrix F, Lombardo E, van Roomen C P, et al. Inhibition of glycosphingolipid synthesis induces a profound reduction of plasma cholesterol and inhibits atherosclerosis development in APOE*3 Leiden and low-density lipoprotein receptor−/− mice. Arterioscler Thromb Vasc Biol 2010; 30:931-7.
Lukina E, Watman N, Avila Arreguin E, et al. Improvement in hematological, visceral, and skeletal manifestations of Gaucher disease type 1 with oral eliglustat tartrate (Genz-112638) treatment: two-year results of a Phase 2 study. Blood 2010.
Lukina E, Watman N, Arreguin E A, et al. A phase 2 study of eliglustat tartrate (Genz-112638), an oral substrate reduction therapy for Gaucher disease type 1. Blood 2010; 116:893-9.
Kroes R A, He H, Emmett M R, et al. Overexpression of ST6GalNAcV, a ganglioside-specific alpha2,6-sialyltransferase, inhibits glioma growth in vivo. Proc Natl Acad Sci USA 2010; 107:12646-51.
Wang X Q, Yan Q, Sun P, et al. Suppression of epidermal growth factor receptor signaling by protein kinase C-alpha activation requires CD82, caveolin-1, and ganglioside. Cancer Res 2007; 67:9986-95.
Lan C C, Liu I H, Fang A H, Wen C H, Wu C S. Hyperglycaemic conditions decrease cultured keratinocyte mobility: implications for impaired wound healing in patients with diabetes. Br J Dermatol 2008.
Spravchikov N, Sizyakov G, Gartsbein M, Accili D, Tennenbaum T, Wertheimer E. Glucose effects on skin keratinocytes: implications for diabetes skin complications. Diabetes 2001; 50:1627-35.
Wang X Q, Sun P, Paller A S. Ganglioside modulation regulates epithelial cell adhesion and spreading via ganglioside-specific effects on signaling. J Biol Chem 2002; 277:40410-9.
Wang X Q, Sun P, Paller A S. Ganglioside GM3 inhibits matrix metalloproteinase-9 activation and disrupts its association with integrin. J Biol Chem 2003; 278:25591-9.
Wang X Q, Sun P, Paller A S. Ganglioside GM3 blocks the activation of epidermal growth factor receptor induced by integrin at specific tyrosine sites. J Biol Chem 2003; 278:48770-8.
Paller A S, Arnsmeier S L, Alvarez-Franco M, Bremer E G. Ganglioside GM3 inhibits the proliferation of cultured keratinocytes. J Invest Dermatol 1993; 100:841-5.
Wang X Q, Sun P, Paller A S. Gangliosides inhibit urokinase-type plasminogen activator (uPA)-dependent squamous carcinoma cell migration by preventing uPA receptor/alphabeta integrin/epidermal growth factor receptor interactions. J Invest Dermatol 2005; 124:839-48.
Kligys K, Claiborne J N, DeBiase P J, et al. The slingshot family of phosphatases mediates Rac1 regulation of cofilin phosphorylation, laminin-332 organization, and motility behavior of keratinocytes. J Biol Chem 2007; 282:32520-8.
Peterburs P, Heering J, Link G, Pfizenmaier K, Olayioye M A, Hausser A. Protein kinase D regulates cell migration by direct phosphorylation of the cofilin phosphatase slingshot 1 like. Cancer Res 2009; 69:5634-8.
Wang Z, Wang M, Carr B I. Involvement of receptor tyrosine phosphatase DEP-1 mediated PI3K-cofilin signaling pathway in sorafenib-induced cytoskeletal rearrangement in hepatoma cells. J Cell Physiol 2010; 224:559-65.
Godsel L M, Dubash A D, Bass-Zubek A E, et al. Plakophilin 2 couples actomyosin remodeling to desmosomal plaque assembly via RhoA. Mol Biol Cell 2010; 21:2844-59.
Kovacs E M, Goodwin M, Ali R G, Paterson A D, Yap A S. Cadherin-directed actin assembly: E-cadherin physically associates with the Arp2/3 complex to direct actin assembly in nascent adhesive contacts. Curr Biol 2002; 12:379-82.
Symons M H, Mitchison T J. Control of actin polymerization in live and permeabilized fibroblasts. J Cell Biol 1991; 114:503-13.
Sehgal B U, DeBiase P J, Matzno S, et al. Integrin beta4 regulates migratory behavior of keratinocytes by determining laminin-332 organization. J Biol Chem 2006; 281:35487-98.
Paller A S, Arnsmeier S L, Chen J D, Woodley D T. Ganglioside GT1b inhibits keratinocyte adhesion and migration on a fibronectin matrix. J Invest Dermatol 1995; 105:237-42.
Karman J, Tedstone J L, Gumlaw N K, et al. Reducing glycosphingolipid biosynthesis in airway cells partially ameliorates disease manifestations in a mouse model of asthma. Int Immunol 2010; 22:593-603.
Prinetti A, Aureli M, Illuzzi G, et al. GM3 synthase overexpression results in reduced cell motility and in caveolin-1 upregulation in human ovarian carcinoma cells. Glycobiology 2010; 20:62-77.
Kabayama K, Sato T, Saito K, et al. Dissociation of the insulin receptor and caveolin-1 complex by ganglioside GM3 in the state of insulin resistance. Proc Natl Acad Sci USA 2007; 104:13678-83.
Salani B, Briatore L, Contini P, et al. IGF-I induced rapid recruitment of integrin beta1 to lipid rafts is Caveolin-1 dependent. Biochem Biophys Res Commun 2009; 380:489-92.
Maggi D, Biedi C, Segat D, Barbero D, Panetta D, Cordera R. IGF-I induces caveolin 1 tyrosine phosphorylation and translocation in the lipid rafts. Biochem Biophys Res Commun 2002; 295:1085-9.
Wang X Q, Sun P, Paller A S. Inhibition of integrin-linked kinase/protein kinase B/Akt signaling: mechanism for ganglioside-induced apoptosis. J Biol Chem 2001; 276:44504-11.
Guilherme A, Torres K, Czech M P. Cross-talk between insulin receptor and integrin alpha5 beta1 signaling pathways. J Biol Chem 1998; 273:22899-903.
Filatov A V, Shmigol I B, Sharonov G V, Feofanov A V, Volkov Y. Direct and indirect antibody-induced TX-100 resistance of cell surface antigens. Immunol Lett 2003; 85:287-95.
Grande-Garcia A, del Pozo M A. Caveolin-1 in cell polarization and directional migration. Eur J Cell Biol 2008; 87:641-7.
Nystrom F H, Chen H, Cong L N, Li Y, Quon M J. Caveolin-1 interacts with the insulin receptor and can differentially modulate insulin signaling in transfected Cos-7 cells and rat adipose cells. Mol Endocrinol 1999; 13:2013-24.
Buckley D A, Loughran G, Murphy G, Fennelly C, O'Connor R. Identification of an IGF-1R kinase regulatory phosphatase using the fission yeast Schizosaccharomyces pombe and a GFP tagged IGF-1R in mammalian cells. Mol Pathol 2002; 55:46-54.
Irvine A D, Sun P, Kos L, Wang X Q, Paller A S. A colorimetric bead-binding assay for detection of intermolecular interactions. Exp Dermatol 2002; 11:462-7.
Couet J, Li S, Okamoto T, Ikezu T, Lisanti M P. Identification of peptide and protein ligands for the caveolin-scaffolding domain. Implications for the interaction of caveolin with caveolae-associated proteins. J Biol Chem 1997; 272:6525-33.
Di W L, Gu Y, Common J E, et al. Connexin interaction patterns in keratinocytes revealed morphologically and by FRET analysis. J Cell Sci 2005; 118:1505-14.
Meng D, Shi X, Jiang B H, Fang J. Insulin-like growth factor-I (IGF-I) induces epidermal growth factor receptor transactivation and cell proliferation through reactive oxygen species. Free Radic Biol Med 2007; 42:1651-60.
Roudabush F L, Pierce K L, Maudsley S, Khan K D, Luttrell L M. Transactivation of the EGF receptor mediates IGF-1-stimulated she phosphorylation and ERK1/2 activation in COS-7 cells. J Biol Chem 2000; 275:22583-9.
Roztocil E, Nicholl S M, Davies M G. Insulin-induced epidermal growth factor activation in vascular smooth muscle cells is ADAM-dependent. Surgery 2008; 144:245-51.
Reinehr R, Sommerfeld A, Haussinger D. Insulin induces swelling-dependent activation of the epidermal growth factor receptor in rat liver. J Biol Chem 2010; 285:25904-12.
Lyu J, Lee K S, Joo C K. Transactivation of EGFR mediates insulin-stimulated ERK1/2 activation and enhanced cell migration in human corneal epithelial cells. Mol Vis 2006; 12:1403-10.
Wang X Q, Sun P, O'Gorman M, Tai T, Paller A S. Epidermal growth factor receptor glycosylation is required for ganglioside GM3 binding and GM3-mediated suppression [correction of suppresion] of activation. Glycobiology 2001; 11:515-22.
Mauvais-Jarvis F, Ueki K, Fruman D A, et al. Reduced expression of the murine p85alpha subunit of phosphoinositide 3-kinase improves insulin signaling and ameliorates diabetes. J Clin Invest 2002; 109:141-9.
Rees R S, Robson M C, Smiell J M, Perry B H. Becaplermin gel in the treatment of pressure ulcers: a phase II randomized, double-blind, placebo-controlled study. Wound Repair Regen 1999; 7:141-7.
Singh-Joy S D, McLain V C. Safety assessment of poloxamers 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, and 407, poloxamer 105 benzoate, and poloxamer 182 dibenzoate as used in cosmetics. Int J Toxicol 2008; 27 Suppl 2:93-128.
Takagi Y, Nakagawa H, Matsuo N, Nomura T, Takizawa M, Imokawa G. Biosynthesis of acylceramide in murine epidermis: characterization by inhibition of glucosylation and deglucosylation, and by substrate specificity. J Invest Dermatol 2004; 122:722-9.
Abe, A, N. S. Radin, J. A Shayman, L. L. Wotring, R E. Zipkin, R Sivakumar, J. M. Ruggieri, K. G. Carson, and B. Ganem. 1995. Structural and stereochemical studies of potent inhibitors of glucosylceramide synthase and tumor cell growth. J Lipid Res. 36:611-21.
Aerts, J. M., R Ottenhoff, A S. Powlson, A Grefhorst, M. van Eijk, P. F. Dubbelhuis, J. Aten, F. Kuipers, M. J. Serlie, T. Wennekes, J. K. Sethi, S. O'Rahilly, and H. S. Overkleeft. 2007. Pharmacological inhibition of glucosylceramide synthase enhances insulin sensitivity. Diabetes. 56:1341-9.
Zhao, H., M. Przybylska, I. H. Wu, J. Zhang, C. Siegel, S. Komarnitsky, N. S. Yew, and S. H. Cheng. 2007. Inhibiting glycosphingolipid synthesis improves glycemic control and insulin sensitivity in animal models of type 2 diabetes. Diabetes. 56:1210-8.
Reiber, G. E., Lipsky, B. A., and Gibbons, G. W. 1998. The burden of diabetic foot ulcers. Am J Surg 176:5S-10S.
Brem, H., and Tomic-Canic, M. 2007. Cellular and molecular basis of wound healing in diabetes. J Clin Invest 117:1219-1222.
Christman, A. L., Selvin, E., Margolis, D. J., Lazarus, G. S., and Garza, L. A. 2011. Hemoglobin Alc Predicts Healing Rate in Diabetic Wounds. J Invest Dermatol.
Guo, S., and Dipietro, L. A. 2010. Factors affecting wound healing. J Dent Res 89:219-229.
Markuson, M., Hanson, D., Anderson, J., Langemo, D., Hunter, S., Thompson, P., Paulson, R., and Rustvang, D. 2009. The relationship between hemoglobin A(1c) values and healing time for lower extremity ulcers in individuals with diabetes. Adv Skin Wound Care 22:365-372.
Marston, W. A. 2006. Risk factors associated with healing chronic diabetic foot ulcers: the importance of hyperglycemia. Ostomy Wound Manage 52:26-28, 30, 32 passim.
Nouvong, A., Hoogwerf, B., Mohler, E., Davis, B., Tajaddini, A., and Medenilla, E. 2009. Evaluation of diabetic foot ulcer healing with hyperspectral imaging of oxyhemoglobin and deoxyhemoglobin. Diabetes Care 32:2056-2061.
Peppa, M., and Vlassara, H. 2005. Advanced glycation end products and diabetic complications: a general overview. Hormones (Athens) 4:28-37.
Acosta, J. B., del Barco, D. G., Vera, D. C., Savigne, W., Lopez-Saura, P., Guillen Nieto, G., and Schultz, G. S. 2008. The pro-inflammatory environment in recalcitrant diabetic foot wounds. Int Wound J 5:530-539.
Aerts, J. M., Ottenhoff, R., Powlson, A. S., Grefhorst, A., van Eijk, M., Dubbelhuis, P. F., Aten, J., Kuipers, F., Serlie, M. J., Wennekes, T., et al. 2007. Pharmacological inhibition of glucosylceramide synthase enhances insulin sensitivity. Diabetes 56:1341-1349.
Zador, I. Z., Deshmukh, G. D., Kunkel, R., Johnson, K., Radin, N. S., and Shayman, J. A. 1993. A role for glycosphingolipid accumulation in the renal hypertrophy of streptozotocin-induced diabetes mellitus. J Clin Invest 91:797-803.
Ando, Y., and Jensen, P. J. 1993. Epidermal growth factor and insulin-like growth factor I enhance keratinocyte migration. J Invest Dermatol 100:633-639.
Usui, M. L., Mansbridge, J. N., Carter, W. G., Fujita, M., and Olerud, J. E. 2008. Keratinocyte migration, proliferation, and differentiation in chronic ulcers from patients with diabetes and normal wounds. J Histochem Cytochem 56:687-696.
Galiano, R. D., Michaels, J., Dobryansky, M., Levine, J. P., and Gurtner, G. C. 2004. Quantitative and reproducible murine model of excisional wound healing. Wound Repair Regen 12:485-492.
Suzuki, K., Olah, G., Modis, K., Coletta, C., Kulp, G., Gero, D., Szoleczky, P., Chang, T., Zhou, Z., Wu, L., et al. 2011. Hydrogen sulfide replacement therapy protects the vascular endothelium in hyperglycemia by preserving mitochondrial function. Proc Natl Acad Sci USA 108:13829-13834.
Ingram, D. A., Lien, I. Z., Mead, L. E., Estes, M., Prater, D. N., Derr-Yellin, E., DiMeglio, L. A., and Haneline, L. S. 2008. In vitro hyperglycemia or a diabetic intrauterine environment reduces neonatal endothelial colony-forming cell numbers and function. Diabetes 57:724-731.
Hashiramoto, A., Mizukami, H., and Yamashita, T. 2006. Ganglioside GM3 promotes cell migration by regulating MAPK and c-Fos/AP-1. Oncogene 25:3948-3955.
Sadagurski, M., Weingarten, G., Rhodes, C. J., White, M. F., and Wertheimer, E. 2005. Insulin receptor substrate 2 plays diverse cell-specific roles in the regulation of glucose transport. J Biol Chem 280:14536-14544.
Lan, C. C., Liu, I. H., Fang, A. H., Wen, C. H., and Wu, C. S. 2008. Hyperglycaemic conditions decrease cultured keratinocyte mobility: implications for impaired wound healing in patients with diabetes. Br J Dermatol.
Spravchikov, N., Sizyakov, G., Gartsbein, M., Accili, D., Tennenbaum, T., and Wertheimer, E. 2001. Glucose effects on skin keratinocytes: implications for diabetes skin complications. Diabetes 50:1627-1635.
Terashi, H., Izumi, K., Deveci, M., Rhodes, L. M., and Marcelo, C. L. 2005. High glucose inhibits human epidermal keratinocyte proliferation for cellular studies on diabetes mellitus. Int Wound J 2:298-304.
Kabayama, K., Sato, T., Kitamura, F., Uemura, S., Kang, B. W., Igarashi, Y., and Inokuchi, J. 2005. TNFalpha-induced insulin resistance in adipocytes as a membrane microdomain disorder: involvement of ganglioside GM3. Glycobiology 15:21-29.
Jeschke, M. G., Schubert, T., and Klein, D. 2004. Exogenous liposomal IGF-I cDNA gene transfer leads to endogenous cellular and physiological responses in an acute wound. Am J Physiol Regul Integr Comp Physiol 286:R958-966.
Jyung, R. W., Mustoe, J. A., Busby, W. H., and Clemmons, D. R. 1994. Increased wound-breaking strength induced by insulin-like growth factor I in combination with insulin-like growth factor binding protein-1. Surgery 115:233-239.
Semenova, E., Koegel, H., Hasse, S., Klatte, J. E., Slonimsky, E., Bilbao, D., Paus, R., Werner, S., and Rosenthal, N. 2008. Overexpression of mIGF-1 in keratinocytes improves wound healing and accelerates hair follicle formation and cycling in mice. Am J Pathol 173:1295-1310.
Liu, J. P., Baker, J., Perkins, A. S., Robertson, E. J., and Efstratiadis, A. 1993. Mice carrying null mutations of the genes encoding insulin-like growth factor I (Igf-1) and type 1 IGF receptor (Igf1r). Cell 75:59-72.
Kabayama, K., Sato, T., Saito, K., Loberto, N., Prinetti, A., Sonnino, S., Kinjo, M., Igarashi, Y., and Inokuchi, J. 2007. Dissociation of the insulin receptor and caveolin-1 complex by ganglioside GM3 in the state of insulin resistance. Proc Natl Acad Sci USA 104:13678-13683.
Karman, J., Tedstone, J. L., Gumlaw, N. K., Zhu, Y., Yew, N., Siegel, C., Guo, S., Siwkowski, A., Ruzek, M., Jiang, C., et al. 2010. Reducing glycosphingolipid biosynthesis in airway cells partially ameliorates disease manifestations in a mouse model of asthma. Int Immunol 22:593-603.
Lukina, E., Watman, N., Avila Arreguin, E., Dragosky, M., Iastrebner, M., Rosenbaum, H., Phillips, M., Pastores, G. M., Kamath, R. S., Rosenthal, D. I., et al. 2010. Improvement in hematological, visceral, and skeletal manifestations of Gaucher disease type 1 with oral eliglustat tartrate (Genz-112638) treatment: two-year results of a Phase 2 study. Blood.
Cox, T. M. 2010. Eliglustat tartrate, an orally active glucocerebroside synthase inhibitor for the potential treatment of Gaucher disease and other lysosomal storage diseases. Curr Opin Investig Drugs 11:1169-1181.
Yew, N. S., Zhao, H., Hong, E. G., Wu, I. H., Przybylska, M., Siegel, C., Shayman, J. A., Arbeeny, C. M., Kim, J. K., Jiang, C., et al. 2010. Increased hepatic insulin action in diet-induced obese mice following inhibition of glucosylceramide synthase. PLoS ONE 5:e11239.
Langeveld, M., Ghauharali, K. J., Sauerwein, H. P., Ackermans, M. T., Groener, J. E., Hollak, C. E., Aerts, J. M., and Serlie, M. J. 2008. Type I Gaucher disease, a glycosphingolipid storage disorder, is associated with insulin resistance. J Clin Endocrinol Metab 93:845-851.
Peterschmitt, M. J., Burke, A., Blankstein, L., Smith, S. E., Puga, A. C., Kramer, W. G., Harris, J. A., Mathews, D., and Bonate, P. L. 2011. Safety, tolerability, and pharmacokinetics of eliglustat tartrate (Genz-112638) after single doses, multiple doses, and food in healthy volunteers. J Clin Pharmacol 51:695-705.
Chujor, C. S., Feingold, K. R., Elias, P. M., and Holleran, W. M. 1998. Glucosylceramide synthase activity in murine epidermis: quantitation, localization, regulation, and requirement for barrier homeostasis. J Lipid Res 39:277-285.
Inoue, M., Fujii, Y., Furukawa, K., Okada, M., Okumura, K., Hayakawa, T., Furukawa, K., and Sugiura, Y. 2002. Refractory skin injury in complex knock-out mice expressing only the GM3 ganglioside. J Biol Chem 277:29881-29888.
Wang, X., Rahman, Z., Sun, P., Meuillet, E., George, D., Bremer, E. G., Al-Qamari, A., and Paller, A. S. 2001. Ganglioside modulates ligand binding to the epidermal growth factor receptor. J Invest Dermatol 116:69-76.
Shirakata, Y., Kimura, R., Nanba, D., Iwamoto, R., Tokumaru, S., Morimoto, C., Yokota, K., Nakamura, M., Sayama, K., Mekada, E., et al. 2005. Heparin-binding EGF-like growth factor accelerates keratinocyte migration and skin wound healing. J Cell Sci 118:2363-2370.
Repertinger, S. K., Campagnaro, E., Fuhrman, J., El-Abaseri, T., Yuspa, S. H., and Hansen, L. A. 2004. EGFR enhances early healing after cutaneous incisional wounding. J Invest Dermatol 123:982-989.
Hamill, K. J., Hopkinson, S. B., Jonkman, M. F., and Jones, J. C. 2011. Type XVII Collagen Regulates Lamellipod Stability, Cell Motility, and Signaling to Rac1 by Targeting Bullous Pemphigoid Antigen 1e to {alpha}6 {beta}4 Integrin. J Biol Chem 286:26768-26780.
Hamill, K. J., Hopkinson, S. B., DeBiase, P., and Jones, J. C. 2009. BPAG1e maintains keratinocyte polarity through beta4 integrin-mediated modulation of Rac1 and cofilin activities. Mol Biol Cell 20:2954-2962.

1-20. (canceled)

21. A method of reducing the level of GM3 in cells comprising administering to said cells a gold nanoparticle (Au NP) conjugated with a small interfering RNA (siRNA) directed against GM3 synthase (GM3S).

22. The method of claim 21, wherein the GM3S siRNA-Au NP is administered to a subject.

23. The method of claim 22, wherein the GM3S siRNA-Au NP is administered topically.

24. The method of claim 23, wherein the GM3S siRNA-Au NP is administered to a wound.

25. The method of claim 24, wherein the subject is diabetic.

26. The method of claim 24, wherein the subject is not diabetic.

27. The method of claim 24, wherein the wound is a cutaneous wound.

28. The method of claim 27, wherein the cutaneous wound is selected from the group consisting of incisions, lacerations, abrasions, puncture wounds, and closed wounds.

29. The method of claim 24, wherein the wound is a chronic cutaneous ulcer.

30. The method of claim 22, further comprising administering an antiseptic, antibiotic, local anesthetic, anti-inflammatory, growth factor, or pain reliever.

31. A composition comprising a gold nanoparticle (Au NP) conjugated with a small interfering RNA (siRNA) directed against GM3 synthase (GM3S).

32. The composition of claim 31, further comprising an antiseptic, antibiotic, local anesthetic, anti-inflammatory, growth factor, or pain reliever.

33. The composition of claim 31, further comprising Aquaphor.
